(19) United States
(12) Patent Application Publication
(10) Pub. No.: US 2026/0128132 A1
(43) Pub. Date: May 7, 2026

(54) Automated Multi-Omics Assay Development System for High-Throughput Proteomic and Metabolomic Quantification

(71) Applicant: Complete Omics International Inc., Baltimore, MD (US)

(72) Inventors: Qing Wang, Baltimore, MD (US); Raghothama Chaerkady, Baltimore, MD (US); Anirudh Kashyap, Baltimore, MD (US); Morgan Fair, Baltimore, MD (US); Liang Zhao, Baltimore, MD (US)

(21) Appl. No.: 19/309,422

(22) Filed: Aug. 25, 2025

Related U.S. Application Data
(60) Provisional application No. 63/686,953, filed on Aug. 26, 2024.
(60) Provisional application No. 63/815,399, filed on May 30, 2025.

(51) Int. Cl. G16B 40/10; G06N 20/00; G16B 40/20; G16H 50/20; G16H 50/70

(52) U.S. Cl. CPC G16B 40/10; G06N 20/00; G16B 40/20; G16H 50/20; G16H 50/70

(57) ABSTRACT

Disclosed are systems and methods for automated chromatographic peak detection and refinement in high-throughput LC-MS/MS datasets, applicable to both proteomics and metabolomics. The invention integrates signal smoothing, apex detection, boundary assignment, and machine learning-based quality scoring into a fully automated pipeline. The system supports multiple acquisition modes (e.g., DIA-PASEF, Orbitrap), chromatographic strategies (C18, C30, HILIC), and biological matrices. Detected peaks are refined using second derivative and percentile-based baseline logic and scored by an XGBoost classifier trained on curated datasets. Quantification-ready outputs are suitable for biomarker panel development, quality control, and diagnostic assay construction. The invention substantially reduces manual curation time while improving reproducibility across samples and platforms.

### CROSS REFERENCE TO RELATED APPLICATIONS

This application claims the benefit of U.S. Provisional Application Nos. 63/686,953 filed Aug. 26, 2024 and U.S. Provisional Application Nos. 63/815,399 filed May 30, 2025. The entire contents of the above-identified applications are hereby fully incorporated herein by reference.

### TECHNICAL FIELD

The invention relates to automated bioinformatics methods for validating and selecting peaks in multi-omics data using supervised machine-learning classifiers. The system processes proteomics, metabolomics, and lipidomics data simultaneously to improve peak picking accuracy and efficiency across diverse biological matrices, particularly blood and plasma samples.

### BACKGROUND

High-throughput liquid chromatography coupled with tandem mass spectrometry (LC-MS/MS) is a cornerstone technology in both proteomics and metabolomics, enabling large-scale detection and quantification of peptides, metabolites, and other small molecules across complex biological samples. Recent advances in data acquisition strategies, such as data-independent acquisition (DIA) and ion mobility separation, have dramatically increased the resolution and dimensionality of mass spectrometry datasets.

Despite these technological advances, the bottleneck in converting raw LC-MS/MS data into reliable, quantification-ready peak boundaries remains a major challenge. In conventional workflows, chromatographic peak identification and refinement are largely dependent on manual curation or semi-automated tools. These approaches are inherently labor-intensive, suffer from subjective interpretation, and lack reproducibility—especially when applied to large-scale datasets involving thousands of molecular targets.

In proteomics, peak picking typically relies on expert evaluation of Skyline outputs or mProphet statistical scoring, while in metabolomics, peak boundaries are often determined using generic software which is not optimized for the unique characteristics of small-molecule elution profiles. Moreover, existing peak detection software often fails to robustly detect low-intensity but biologically meaningful peaks, leading to a loss of sensitivity in downstream biomarker validation (Coyle et al., J Proteome Res. 2025 Jan. 3;24(1):244-255.).

This inefficiency presents a critical barrier to the clinical and translational application of omics-based biomarker panels, where consistency, throughput, and reproducibility are paramount. For example, biomarker panel development for diagnostic assays requires reproducible identification of the characteristic chromatographic feature across hundreds of patient samples and multiple batches. Manual inspection is not scalable in such settings and contributes to prolonged development timelines.

Therefore, there exists a need for an automated, scalable, and accurate peak-picking solution that eliminates manual intervention, harmonizes boundary assignment across runs, and maintains sensitivity for low-abundance species. The present invention addresses this unmet need by introducing a dual-omics pipeline that leverages machine learning and signal processing techniques tailored for both proteomics and metabolomics datasets. No prior approach (i) jointly optimizes proteomic and metabolomic peak selection under one pipeline, (ii) harmonizes boundaries across runs using apex-anchored offsets, and (iii) leverages reproducible blood-matrix patterns as endogenous landmarks.

### SUMMARY

In one aspect, the present disclosure provides a method for automated peak picking in multi-omics biological sample data. The method comprises preprocessing biological sample data to generate feature files containing candidate peaks for target analytes, extracting features from said feature files including coelution metrics, spectral similarity measures, signal quality parameters, and intensity values, training a machine learning model on manually curated data to predict high-quality peaks, applying the trained model to new datasets to predict peak quality for each candidate peak, and postprocessing predictions to retain the most reliable peaks for each target analyte.

In some embodiments, the biological sample data comprises plasma proteomics, metabolomics, and lipidomics data analyzed simultaneously. The machine learning model comprises an XGBoost gradient boosting framework optimized through cross-validation, wherein features extracted include coelution count, dot product score, signal-to-noise ratio, shape correlation, and peak intensity.

In proteomics applications, the method processes Selected Reaction Monitoring (SRM) or Multiple Reaction Monitoring (MRM) data with mProphet feature file generation, wherein coelution metrics comprise counting transitions that coelute within defined retention time windows, and spectral similarity measures comprise dot product calculations comparing observed peaks to reference library spectra.

In metabolomics applications, the method further comprises smoothing intensity signals using Savitzky-Golay filtering, computing second derivatives of smoothed intensity data, identifying zero-crossings of the second derivative to define peak boundaries, and applying background correction and width standardization using interquartile range methods. Peak boundaries are adjusted using extension factors to account for peak asymmetry.

In certain embodiments, the biological sample data comprises blood or plasma samples, and the method utilizes consistent noise patterns from blood matrix components as reproducible landmarks for enhanced peak identification without requiring exogenous internal standards. The noise patterns originate from high-abundance proteins, lipids, or other prevalent blood substances that provide consistent reproducibility across different specimens.

The method further comprises normalizing peak area data using Multi-point Normalized Protein eXpression (MNPX) values, wherein normalization is performed separately for different analytical clusters based on peak intensities, hydrophobicities, and retention times. Multiple reference points are used for normalization, tailored to each cluster of target analysis.

In some embodiments, the manually curated data comprises samples from patients with cancer, cardiovascular disease, neurodegenerative disease, or autoimmune disease for training disease-specific pattern recognition. The target analytes comprise proteins, metabolites, and lipids simultaneously analyzed from the same biological sample for detecting disease-specific molecular signatures.

In another aspect, the present disclosure provides a system for automated peak picking in multi-omics data comprising a data preprocessing module configured to generate feature files from biological sample data, a feature extraction module configured to extract and label analytical features, a machine learning module configured to train a predictive model using extracted features and manual validation labels, and a postprocessing module configured to select highest-scoring predictions for each target analyte.

In some embodiments, the machine learning module implements XGBoost with hyperparameters optimized through grid search and cross-validation, and the system further comprises a normalization module configured to apply Multi-point Normalized Protein eXpression (MNPX) values. The peak analysis components may be implemented as interoperable modules and containerized for scalable, cloud-based processing.

The method includes quality control parameters wherein reliable detection requires a signal-to-noise ratio of at least 1.5, retention time exceeding 1.5 minutes, and background signals at least one order of magnitude lower than analyte peak intensity. Fragment ion ratios between transitions remain within ±20-30% of reference values and spectral similarity scores exceed 0.5-0.6 for validation.

In another aspect, the present disclosure provides a method of disease diagnosis comprising obtaining a blood or plasma sample from a subject, analyzing said sample using the automated peak picking method to identify molecular signatures comprising proteins, metabolites, and lipids, comparing identified signatures to disease-specific reference patterns, and determining disease status based on signature comparison.

In some embodiments, the molecular signatures are associated with cancer, cardiovascular disease, neurodegenerative disease, or autoimmune disease, and the analysis provides simultaneous multi-omics characterization from a single sample. The method may be applied for pharmaceutical quality control, biological product release testing, or the development of disease-relevant biomarker panels.

In some embodiments, the disclosed method is implemented as a non-transitory computer-readable medium comprising instructions that, when executed, cause a computing system to perform any of the disclosed steps. The system may further include a biomarker database and a report generation module for outputting peak quality and abundance information.

### BRIEF DESCRIPTION OF THE DRAWINGS

FIG. 1 illustrates a unified pipeline for high-throughput biomarker panel development from both proteomic and metabolomic data sources, integrating mass spectrometry-based acquisition, targeted assay development, and machine-learning-driven peak picking. In the proteomics workflow (FIG. 1A), a timsTOF HT mass spectrometer performs data-independent acquisition (DIA-PASEF), generating over 150,000 peptide-spectrum matches (PSMs) per sample. The most abundant proteotypic peptide for each protein is automatically selected and directed to a targeted assay development module, where transitions are scheduled for QqQ-based quantification. Peak quality is evaluated using an XGBoost classifier trained on manually reviewed chromatograms, which considers features such as co-elution count, peak shape, and retention time consistency. In the metabolomics workflow (FIG. 1B), a Q Exactive mass spectrometer is used with multiple chromatographic separations (C18, C30, and HILIC) to capture a broad range of metabolites. Raw traces are processed through a peak picking engine that applies Savitzky-Golay filtering, detects apexes within a retention time window, and assigns peak boundaries using second-derivative zero-crossings and percentile-based baseline extension. Both workflows feed into the generation of a validated biomarker panel consisting of high-quality peptides and metabolites suitable for reproducible quantification, highlighting the system's scalability, modularity, and automation in transforming discovery-phase LC-MS/MS data into clinically actionable outputs within a rapid turnaround time.

FIG. 2 (FIG. 2) illustrates a graphical user interface (GUI) that represents the metabolomics peak picking pipeline and the functional workflow of the peak picker module used for automated boundary detection in LC-MS/MS chromatographic data. The interface allows users to select a sample file (e.g., Sample 001.mzXML) and a target molecule (e.g., PC(16:0/18:2)) with a known or predicted retention time. Users can configure processing parameters, including the threshold for boundary extension (expressed as a fraction of the apex intensity) and the maximum allowed extension in data points. The central panel displays the extracted ion chromatogram for the selected analyte, showing the apex and automatically determined start and end points using signal intensity profiles. Detected peaks are summarized with metadata such as start time, end time, apex time, and peak width. The tool also allows batch processing of multiple molecules across samples, as shown in the processed results table, and supports data import and export functions for streamlined integration with external workflows. This module automates key aspects of peak curation, ensuring reproducibility and reducing manual error in metabolite quantification.

FIG. 3 (FIG. 3) shows the Complete360 Peak Picker workflow for proteomics data processing, which enables fully automated chromatographic peak refinement based on Skyline output directories. The interface features a three-step modular process: (1) The Peak Picking Model accepts user-uploaded Skyline directories and performs automatic peak detection using a trained XGBoost machine learning model. This model evaluates features extracted from the raw chromatographic traces, such as co-elution profiles, peak shape, and signal-to-noise characteristics, to select the most representative peak. The output of this module is the set of best peak boudaries leveraging the XGBoost pipeline. (2) The RT Alignment Module then takes the most abundant peak, defined as the one with the largest area, identified in each file and aligns retention times across all runs. The alignment uses these high-confidence peaks to correct for systematic drift. In this workflow, retention time coefficients of variation (CVs) are typically less than 0.5 percent, with observed drifts between 0.2 and 0.5 minutes. The aligned retention time of the most abundant peak is used to adjust boundary definitions across all samples. (3) The Dynamic RT Adjustment Module uses the apex retention time values from all aligned samples within the defined retention window to recalculate peak boundaries. It anchors the peak width using the boundary span from the most abundant reference sample and applies this consistent offset, left and right of the apex, to all other samples. This ensures uniform peak integration across samples despite minor chromatographic shifts.

FIG. 4 (FIG. 4) shows a radial plot of feature importance values derived from an XGBoost classifier trained to distinguish high-quality from low-quality chromatographic peaks. Each axis represents a predictive feature used by the model, including co-elution count, signal intensity, signal-to-noise ratio, peak shape, dot-product similarity to reference spectra, and library intensity variance. The magnitude along each axis reflects the relative contribution of that feature to the model's decision-making process. In this visualization, co-elution was the highest-ranked feature in representative models, indicating that the simultaneous elution of related transitions is a key determinant of peak quality. Other features contribute modestly, with peak intensity and shape also showing importance. This figure highlights the model's ability to replicate human expert judgment by prioritizing chromatographic features consistent with high-confidence peak detection.

FIG. 5 (FIG. 5) illustrates a machine learning pipeline for automated chromatographic peak classification using XGBoost. The proteomics workflow begins with manually labeled training data, where chromatograms are annotated as either correct or incorrect based on peak shape and retention time characteristics. From these chromatograms, nine key features are extracted, including Peak Apex RT, Peak Symmetry, Peak Kurtosis, Signal-to-Noise Ratio, Background Intensity, Intensity, Dot-Product Score, and Co-elution Score. These features are used to train an XGBoost classifier, which learns to distinguish between true and false peaks. The trained model is then validated on an independent test set of chromatograms, achieving a high performance with an area under the ROC curve (AUC) of 0.95 and a test accuracy of 93%, demonstrating the model's effectiveness in accurately classifying chromatographic features.

FIG. 6 Complete360 multi-omics workflow and comprehensive target characterization platform for blood-based molecular diagnostics. FIG. 6A shows the schematic overview of the Complete360 pipeline, starting from blood sample collection, progressing through low-abundance protein enrichment using chemical-biological depletion methods, enzymatic digestion to peptides, and mass spectrometry analysis, with resulting data processed through the CompletePeaking software suite employing XGBoost machine learning algorithms for automated peak picking across proteomics, metabolomics, and lipidomics datasets, leading to diagnostic report generation and integrating CompleteBank-Discovered containing over 17,000 proteins and 2,900 metabolites/lipids and CompleteBank-Validated with optimized detection parameters for 10,598 proteins and 2,157 small molecules supporting disease diagnostic panel development across cardiovascular, oncology, neurological, and immune disorders. FIG. 6B illustrates the distribution of molecular targets across various tissues and disease-relevant organs including brain, liver, cervix carcinoma, lung, erythroleukemia, placenta, kidney, and other major human tissues, demonstrating the platform's capability to detect tissue-specific proteins circulating in blood for comprehensive molecular phenotyping and disease-specific biomarker identification. FIG. 6C shows comprehensive molecular target characterization with subcellular protein localization analysis revealing 34.5% cytoplasmic, 11.8% secreted, 9% cytoskeletal, 8.2% mitochondrial, 8.0% endoplasmic reticulum, 7.8% cell projection, and 6.5% Golgi apparatus proteins, metabolite analysis encompassing 762 polar compounds including organic acids, amino acids, nucleotides, and carbohydrates covering major human metabolic pathways, and lipid analysis including 1,395 species across more than 24 subclasses including phospholipids, triglycerides, sphingolipids, and eicosanoids, enabling simultaneous multi-omics characterization from single biological samples.

FIG. 7 Analytical performance capabilities of Complete360 platform for protein quantification demonstrating unprecedented dynamic range and reproducibility in plasma samples. FIG. 7A shows the dynamic range of plasma protein concentrations detectable by the Complete360 platform spanning from approximately 3.5 pg/mL to over 100 μg/mL, surpassing traditional mass spectrometry detection limits and capturing the vast majority of physiologically relevant plasma proteins, with demonstrated ability to detect extremely low-abundance biomarkers critical for early disease detection, including proteins such as Isocitrate dehydrogenase subunit beta detected at 8.3 pg/mL concentration with robust signal intensity sufficient for 1:1000 sample dilution analysis. FIG. 7B demonstrates quantification reproducibility analysis showing the relationship between plasma protein concentration and measurement precision, where higher protein concentrations are associated with lower coefficients of variation (CV), with the platform achieving remarkable precision with average CV of 3.92% for 36 proteins spanning six orders of magnitude concentration range and a strong inverse correlation (R²=0.51) between mass spectrometry signal intensity and CV, with 4,361 proteins exhibiting CVs below 10% and median CV of 4.77%, meeting stringent clinical diagnostic requirements for biomarker quantification.

FIG. 8 Disease-associated molecular changes revealed through Complete360 multi-omics analysis across multiple pathological conditions enabling simultaneous detection of disease-specific protein, metabolite, and lipid signatures from single plasma samples. FIG. 8A shows comprehensive protein quantification analysis processing 10,598 plasma proteins from individuals with breast cancer (BRCA), colon cancer (CRC), lung adenocarcinoma (LUAD), ovarian cancer (OVC), pancreatic adenocarcinoma (PDAC), prostate adenocarcinoma (PRAD), Alzheimer's disease, ulcerative colitis, and healthy controls, demonstrating that majority of tumor tissue proteins from CPTAC studies could be quantitatively analyzed in corresponding patient plasma samples including 7,825 BRCA, 7,224 PRAD, 7,599 PDAC, 7,110 OVC, 8,047 LUAD, and 6,445 CRC tumor-associated proteins detectable in circulation. FIG. 8B reveals over-expressed proteins showing at least 2-fold elevation in different cancer types with 1,834 BRCA, 1,418 CRC, 1,389 LUAD, 1,178 OVC, 1,211 PDAC, and 1,872 PRAD tumor-associated proteins detected in plasma samples, demonstrating the platform's sensitivity for detecting disease-associated molecular changes in circulation. FIG. 8C shows molecular pathway analysis using Hallmark gene signature analysis identifying both common pathways including apical junction, epithelial-mesenchymal transition, mTORC1 signaling, p53 pathway, and glycolysis, as well as disease-specific molecular programs including adipogenesis and complement for BRCA, xenobiotic metabolism and DNA repair for CRC, TNF-alpha signaling and oxidative phosphorylation for LUAD, myogenesis and mitotic spindle for OVC, DNA repair and allograft rejection for PDAC, and adipogenesis, coagulation, and heme metabolism for PRAD. FIG. 8D demonstrates cancer-type specific protein identification revealing unique molecular signatures with 517 BRCA, 174 CRC, 255 LUAD, 142 OVC, 154 PDAC, and 552 PRAD proteins showing cancer-specific elevation patterns for disease-specific biomarker identification and differential diagnosis applications. FIG. 8E shows metabolomics analysis using the Complete360-MyMeta pipeline quantifying 762 polar metabolites across various compound classes, revealing disease-specific elevation patterns with at least 2-fold increases compared to normal controls, providing complementary molecular information enhancing diagnostic accuracy. FIG. 8F illustrates disease-type specific metabolite identification revealing unique small molecule signatures demonstrating both disease-shared and disease-specific metabolite changes, indicating that multi-omics integration provides enhanced diagnostic precision compared to single-analyte class approaches.

FIG. 9 Breast cancer-associated molecular changes revealed through Complete360 platform for disease-specific biomarker discovery and validation demonstrating robust disease signature detection with cross-platform reproducibility and pathway-level insights. FIG. 9A shows principal component analysis (PCA) of breast cancer plasma proteomic profiles from the Evelyn cohort with clear separation between breast cancer (BC) and non-cancer (NORM) samples with distinct cluster formation, indicating robust disease-associated proteomic signatures with all cancer samples differentiated from non-cancerous controls. FIG. 9B demonstrates independent validation using the Amelia analytical platform showing PCA analysis with clear separation between disease and control groups, confirming reproducibility and reliability of Complete360 breast cancer signatures across different mass spectrometry instruments. FIG. 9C illustrates cross-platform correlation analysis comparing log2 fold changes between Amelia and Evelyn cohorts showing consistency with correlation coefficient of 0.54, with red-highlighted significantly upregulated biomarkers including THBS1, TUBA4A, TPM3, PACSIN2, and SOD1, validating that 105 of 331 initially identified elevated proteins were reproducibly detected across different Complete360 instruments. FIG. 9D shows single-sample gene set enrichment analysis (ssGSEA) revealing activation of cancer-relevant pathways including TGF-beta signaling, reactive oxygen species pathway, IL-6/JAK/STAT3 signaling, complement, and Myc targets for elevated proteins, while downregulated proteins show enrichment in estrogen response and interferon alpha pathways. FIG. 9E demonstrates Vinculin (VCL) expression analysis showing significant elevation in breast cancer plasma samples involved in cell migration and metastasis with robust signal detection and clear disease-associated elevation pattern. FIG. 9F shows Ficolin-2 (FCN2) protein expression analysis revealing significant upregulation in breast cancer plasma samples, highlighting detection of immune system proteins playing roles in immune surveillance and antitumor responses. FIG. 9G illustrates Ribosomal protein L22 (RPL22) expression analysis showing significant elevation in breast cancer plasma samples, demonstrating sensitivity for detecting proteins frequently deregulated in cancer biology with clear disease-associated expression patterns supporting potential biomarker utility.

FIG. 10 Complete360-based pQTL analysis for demographic and physiological trait associations demonstrating population-scale proteomic analysis capabilities revealing systematic protein expression associations with age, sex, and BMI for personalized medicine applications. FIG. 10A shows age-related proteomic variation analysis using volcano plot visualization revealing multiple proteins involved in inflammation, extracellular matrix remodeling, and cellular senescence displaying significant age-associated expression changes, demonstrating the platform's utility for capturing dynamic physiological aging markers and developing biological clock signatures supporting health monitoring and age-related disease risk assessment applications. FIG. 10B illustrates sex-based proteomic differences analysis showing distinct regulation patterns in proteins related to hormonal signaling, immune response, and lipid metabolism, providing insights into biological differences underlying sex-specific disease susceptibility, treatment response variations, and molecular basis for personalized therapeutic approaches accounting for biological sex differences. FIG. 10C demonstrates BMI-associated proteomic changes analysis identifying key metabolic regulators including TRIB3 (obesity and insulin resistance regulator), INHBE (fat distribution determinant), and ERBB4 (brain-regulated energy expenditure modulator) showing strong BMI-correlated expression profiles, with LEP (leptin) emerging as primary biomarker for adiposity and metabolic regulation, demonstrating platform precision for metabolic disease risk stratification and personalized health monitoring applications.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2nd edition 2013 (E. A. Greenfield ed.); Animal Cell Culture (1987) (R. I. Freshney, ed.); Benjamin Lewin, Genes IX, published by Jones and Bartlet, 2008(ISBN 0763752223); Kendrew et al. (eds.), The Encyclopedia of Molecular Biology, published by 10Blackwell Science Ltd., 1994 (ISBN 0632021829); Robert A. Meyers (ed.), Molecular Biologyand Biotechnology: a Comprehensive Desk Reference, published by VCH Publishers, Inc., 1995(ISBN 9780471185710); Singleton et al., Dictionary of Microbiology and Molecular Biology 2nded., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which themodifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### System Overview and Workflow Architecture

The present disclosure relates to automated systems and methods for high-throughput chromatographic peak detection and refinement in LC-MS/MS-based proteomic and metabolomic workflows. Traditional methods for identifying, validating, and quantifying chromatographic peaks are time-consuming, heavily reliant on expert curation, and prone to variability. These limitations hinder reproducibility, reduce throughput, and delay the translation of discovery-phase data into clinically actionable assays (Gillet et al., Mol Cell Proteomics. 2012 Jun.;11(6): O111.016717.).

To overcome these challenges, the present invention provides a fully automated software pipeline that integrates signal processing and machine learning for real-time chromatographic peak assessment. The solution is applicable across both proteomics and metabolomics, accommodating diverse acquisition modes such as DIA-PASEF and Orbitrap-based LC-MS/MS. Key features include automated smoothing, apex detection, boundary refinement, and quality scoring using a machine learning classifier trained on manually curated datasets. The system outputs quantification-ready features suitable for targeted QqQ assay generation, dramatically reducing assay development time from days or weeks to several minutes.

Unlike existing tools such as Skyline (MacLean et al., Bioinformatics. 2010 Apr. 1;26(7):966-8.) and mProphet (Reiter et al., Nat Methods. 2011 May;8(5):430-5.), which rely on hand-curated peak boundaries or rigid statistical models, the present invention uses a generalizable set of features derived from the raw chromatographic profile. These include co-elution count, dot-product score to reference spectra, signal-to-noise ratio, and retention time variance. An XGBoost model trained on manually labeled chromatograms serves as the core classifier, providing peak quality scores that replicate expert review. The architecture modularly separates detection, scoring, and post-processing, which enables seamless integration into multi-omics pipelines and compatibility with diverse data formats (e.g., mzXML, mzML, Skyline reports).

This approach offers several technical advantages: (1) significant reduction in manual curation workload; (2) enhanced consistency across samples and analytical batches; (3) scalability to thousands of peptides and metabolites per dataset; (4) adaptability to varying chromatographic and instrument conditions; and (5) accurate detection of low-abundance features that are often missed in threshold-based workflows. Together, these capabilities provide a robust and reproducible framework for biomarker panel development in both research and clinical settings.

High-throughput LC-MS/MS platforms using DIA/DDA for proteomics (Meier et al., Mol Cell Proteomics. 2018 Dec.;17(12):2534-2545.) and full-scan Orbitrap-based metabolomics (e.g., Q Exactive) enable broad, untargeted data acquisition during the discovery phase. However, the ultimate utility of this method lies in their translation into targeted, quantifiable assays for clinical and industrial applications. Manual curation of chromatographic peaks is impractical at scale and prone to variability that compromises reproducibility. The present disclosure addresses this challenge by providing a fully automated system for peak detection, boundary harmonization, and transition selection, specifically designed to generate quantification-ready outputs for targeted methods such as MRM or PRM. By combining signal processing and machine learning, the dual omics pipeline ensures consistent and high-confidence peak definitions, enabling rapid deployment of targeted assays derived from high-throughput discovery data.

As used herein, a “chromatographic peak” refers to a contiguous region of signal intensity in a chromatographic time series corresponding to the elution of an analyte. A “retention time window” is a predefined region surrounding the expected retention time of an analyte used to guide apex detection. A “quantification-ready peak” includes apex, start, and end time information and passes quality filters defined by classifier output or signal thresholds. “Co-elution count” refers to the number of simultaneous eluting species within the peak window, which serves as a proxy for signal confidence. A “classifier” refers to a trained machine learning model (e.g., XGBoost) that outputs a binary or probabilistic quality assessment.

“Apex-anchored offset” means the left/right time differences between a peak's apex and its start/end boundaries, computed in a reference instance and applied to other instances for harmonized boundary assignment.

“Endogenous landmark” means a reproducible background feature present in blood/plasma matrices used to index retention time and validate candidate peaks.

“Boundary harmonization” means applying apex-anchored offsets to generate consistent start/end boundaries across samples or runs.

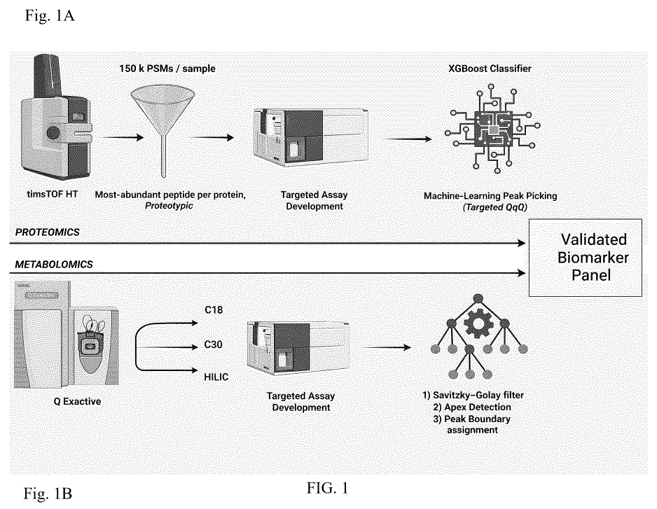

The system is schematically illustrated in FIG. 1. In the proteomic branch, data is acquired on a timsTOF HT instrument operating in DIA-PASEF mode. This results in over 150,000 peptide-spectrum matches (PSMs) per sample, from which proteotypic peptides are identified for downstream targeted assays. In the metabolomic branch, samples are run on an Orbitrap Q Exactive using orthogonal LC chemistries including C18, C30, and HILIC columns to maximize coverage across hydrophobic and polar compound classes.

Raw chromatographic traces are smoothed using a Savitzky-Golay filter, a method known to preserve peak shape while reducing baseline noise (Savitzky & Golay, Analytical Chem. 1964. Vol 36/Issue 8). Apex detection is performed within a ±1-minute window around the expected retention time for each analyte. The initial boundaries of each candidate peak are defined by locating zero-crossings in the second derivative of the smoothed intensity signal, which correspond to inflection points flanking the apex. These boundaries are then extended until the signal drops to a baseline level estimated as 5% above the local background. The local baseline is calculated using the 20th percentile of intensity values within a symmetric window of ±50 data points centered around the apex. A maximum extension limit of 50 data points is applied on either side to prevent boundary overreach. These parameters are fully configurable but empirically validated to provide accurate results across both peptide and metabolite chromatograms.

In some embodiments, the baseline threshold is derived dynamically for each chromatographic region. For example, the 20th-percentile value is computed within a ±50 scan window surrounding the apex, a window size selected based on empirical evaluation of boundary behavior across metabolomic targets in lipid panels and polar metabolite panels. This percentile-based approach minimizes influence from spurious noise spikes and tailing effects in complex elution profiles, thereby improving robustness of the lower boundary estimate. This refinement enables consistent performance across low- and high-intensity features, supporting the reproducibility needed for clinical or industrial settings.

### Proteomics Peak Detection System

As used herein in the context of proteomic analysis, a “peptide-spectrum match (PSM)” refers to a computational association between an observed tandem mass spectrum and a theoretical peptide sequence derived from in silico digestion, typically assigned in DIA-based workflows. A “Skyline report” refers to a tab-delimited export file produced by the Skyline software platform, containing peptide and transition-level chromatographic features such as retention time, peak area, dot-product similarity, and peak boundaries. An “mProphet score” refers to a probabilistic confidence metric generated by the mProphet statistical modeling algorithm, which distinguishes true peptide signals from decoy-based false positives based on features such as dotp, mass error, and co-elution. “Co-elution count” refers to the number of transitions or fragment ions that elute within a defined retention time window, providing internal confirmation of peptide identity. “Dot-product similarity” refers to the cosine similarity between observed and expected transition intensity profiles for a peptide, used to assess peak quality. “Boundary harmonization” refers to the process of aligning or adjusting start and end retention times across replicate files or experimental conditions to improve the reproducibility and comparability of integrated peak areas. A “quantification-ready peak” refers to a peptide feature for which start and end boundaries, apex retention time, and a classifier-assigned quality score are defined, and which meets criteria sufficient to be used in scheduled MRM/PRM quantification or statistical comparisons across conditions.

Proteomic biomarker discovery often begins with data-independent acquisition (DIA) LC-MS/MS experiments using high-resolution instruments such as the timsTOF HT. These workflows produce over 150,000 peptide-spectrum matches (PSMs) per sample, representing a complex landscape of candidate targets for downstream quantification. Manual curation of this data to select quantification-ready peaks is time-intensive and poorly scalable. Moreover, expert bias and lack of standardization often lead to inconsistent or suboptimal assay development.

The present invention addresses these limitations by providing a machine learning-guided, automated pipeline for peptide-level peak detection, boundary refinement, and retention time alignment. The system supports DIA-PASEF data and takes as input pre-processed Skyline reports and mProphet feature files. Candidate peptides are ranked and selected based on reproducibility, co-elution behavior, and predicted peak quality across files. The final output is a set of harmonized, high-confidence peptide peaks suitable for direct transition into QqQ assays.

In some embodiments, the input pipeline begins by generating and cleaning mProphet and Skyline reports using custom scripts. The combined report is then labeled using a supervised strategy that assigns “good” or “bad” peak labels based on manually curated retention time alignments, peak shape descriptors, and co-elution metrics. Labeling is performed using a labeling module that applies reproducibility and peak symmetry thresholds to create a binary classification for each peptide feature. This labeled dataset serves as ground truth for downstream model training.

In some embodiments, the classifier is implemented as an XGBoost gradient-boosted decision tree, trained on over 3,000 labeled peptide peaks spanning multiple sample types and acquisition conditions. Features include intensity variance, dot-product similarity variance, co-elution count, signal-to-noise ratio, and shape asymmetry. Model hyperparameters such as tree depth, learning rate, gamma, and regularization are tuned using grid search cross-validation to maximize area under the ROC curve (AUROC). The final model is saved as a persistent model file and used during prediction to score candidate peaks on a 0-1 scale. Training may address class imbalance using weighted losses (e.g., scale_pos_weight) or stratified sampling.

In some embodiments, peak prediction is performed using a classifier module that accepts a trained model and a peptide-level feature matrix. The algorithm scores each candidate peak per peptide and retains the one with the highest score per sample. Peaks that fall below a quality threshold are excluded. The retained peaks are stored in a CSV output, which includes the start and end retention times, apex retention time, peptide-modified sequence, and file name.

In some embodiments, the system includes a boundary harmonization module for intra-run and inter-run consistency. For intra-sample alignment, a boundary propagation module identifies the most intense replicate instance of each peptide and copies its start and end boundaries to all other technical replicates of the same peptide. This ensures that all peptides within a run use a uniform peak window for quantification. For inter-sample harmonization, a retention alignment module identifies the most intense global instance of a peptide and computes average offsets between its apex and each boundary. These apex-relative offsets are then applied across all samples, preserving peak shape while normalizing boundaries across retention time drift.

In some embodiments, the software pipeline includes the following modules:
a. A labeling module, functionally corresponding to a script that processes merged Skyline and mProphet reports and applies manual labeling criteria (e.g., peak symmetry, reproducibility, and co-elution). The module assigns binary “good” or “bad” labels and outputs a labeled dataset for classifier training. Labeling logic incorporates empirical thresholds and visual inspection of peak shapes using known standards and replicates.
b. A prediction module, which accepts the trained XGBoost model and applies it to a peptide feature table. It scores all candidate peaks and selects, for each peptide per file, the candidate with the highest quality score. Peaks below a confidence threshold are discarded. The module outputs structured results containing apex time, start and end boundaries, file name, and peptide ID.
c. A stationary boundary assignment module, which propagates the retention boundaries of the highest-confidence replicate to all technical replicates within the same run. The module ensures that all instances of a peptide are quantified over the same time window, minimizing intra-batch variability.
d. An alignment module, which computes global average boundary offsets relative to the apex of the highest-quality instance across all samples. These average offsets are then applied to each local apex to generate harmonized boundaries across the dataset. The approach reduces retention variation caused by chromatographic drift.
e. A reporting module, which calculates average apex retention times across all samples for each peptide and stores these values for transition scheduling and method reproducibility. These values are used in downstream QqQ method development and serve as internal reference standards.
In some embodiments, boundary harmonization is performed in two stages. First, the system selects the most intense peptide instance in each file and propagates its start and end boundaries to all other replicates of the same peptide. This ensures within-file consistency for quantification. Next, the system aggregates all files to identify the most intense global peak per peptide and computes average left/right offsets from its apex. These offsets are then applied to all samples using apex-relative alignment, producing normalized peak boundaries across files even when retention time drift is present. If ion-ratio checks or spectral similarity fail in a given run, harmonized boundaries may be locally adjusted to exclude interference while preserving apex-anchored width.

In some embodiments, the system computes average retention times for each peptide across multiple files. This average RT is used for transition scheduling in MRM development or to identify retention drift across experiments.

Final outputs include structured CSV files containing file name, peptide-modified sequence, start and end retention times, apex time, and optionally total area or quality score. These outputs are compatible with scheduled MRM/PRM assay generation tools and laboratory LIMS systems. In benchmarking tests, the automated pipeline reduced manual review time by over 90% and improved peak boundary reproducibility across replicates by more than 30% relative to expert-curated results.

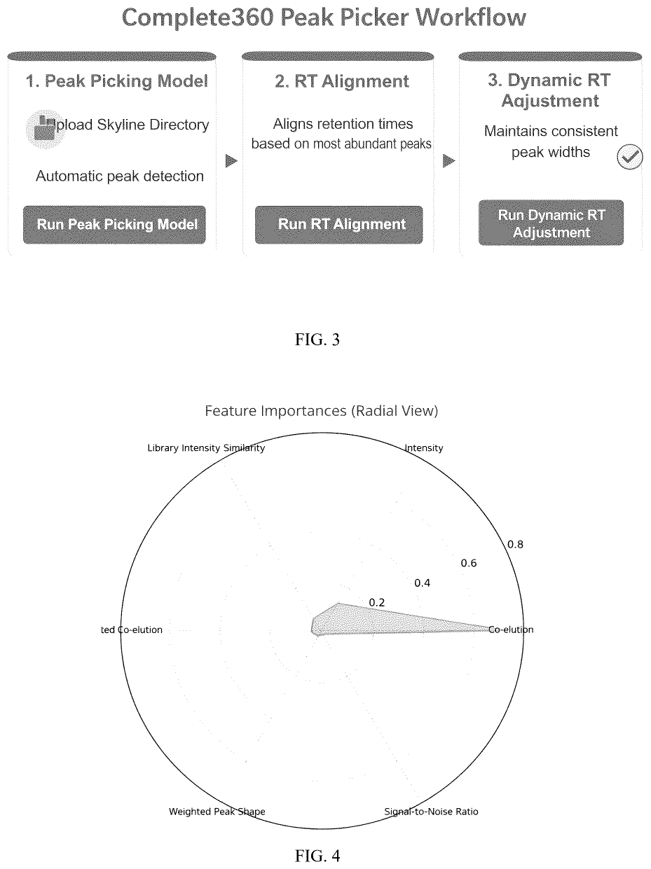

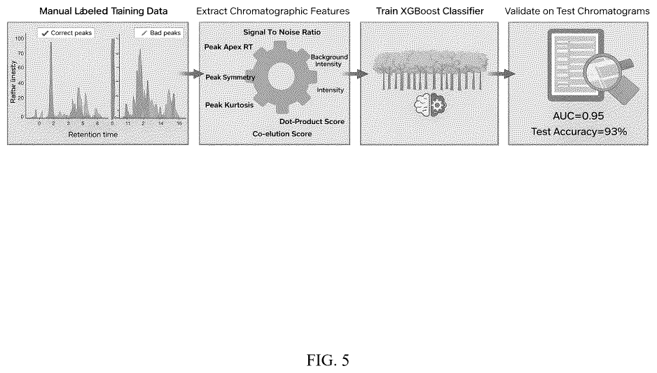

In some embodiments, feature importance analysis of the trained model revealed that co-elution count was the most influential variable in peak quality prediction, followed by dot-product similarity and intensity variance. These findings match human heuristics and support the model's alignment with expert judgment. FIG. 3 (Complete360 Proteomics Peak Picker) illustrates this three-step workflow: (1) detection from Skyline directory, (2) retention time alignment, and (3) dynamic RT correction to maintain consistent peak widths.

### Metabolomics Peak Detection System

As used herein in the context of metabolomic analysis, “explicit retention time” refers to a user-defined or model-derived elution time used as a starting point for peak search. “Quantification-ready peak” refers to a metabolite feature for which apex retention time, start and end boundaries, and file-level identifiers are determined with quality suitable for downstream quantification. “Baseline extension” refers to the process of extending candidate peak boundaries until the smoothed signal drops to a threshold level defined as a fixed fraction above the local baseline. “Second derivative zero-crossing” refers to the inflection points in the smoothed signal profile where the second derivative changes sign, marking the approximate peak start and end. A “fallback mechanism” refers to the automatic expansion of the search window to the full chromatogram if no peak is detected near the expected retention time. “Fraction of apex” refers to the signal intensity threshold, typically defined as a percentage (e.g., 5%) of the apex intensity, used in the boundary extension procedure.

Metabolomics LC-MS/MS datasets pose unique challenges due to a broader range of chemical diversity, variable ionization behavior, and a wide distribution of chromatographic peak shapes. Traditional proteomics-oriented peak picking tools often fail to accommodate these complexities, leading to incomplete or inconsistent quantification. In high-throughput metabolomics studies, manual peak annotation is infeasible, and the lack of scalable and reproducible boundary detection algorithms limits quantitative accuracy and inter-study comparability.

The present invention addresses these limitations by implementing a dedicated, fully automated software suite optimized for metabolomics data. This suite reads raw intensity profiles from structured CSV input, performs retention time-anchored peak detection, and applies a two-stage boundary assignment algorithm combining second derivative and percentile-based signal analysis. The output is a harmonized, quantification-ready peak boundary table directly compatible with statistical and downstream reporting tools.

In some embodiments, the system reads input from a CSV table comprising comma-separated fields: Times, Intensities, Molecule, Explicit RT, and File Name. Each row corresponds to a unique sample-metabolite pair and contains chromatographic trace vectors as strings. A parsing module reads and validates this input using structured iteration and strict data integrity checks. Rows missing retention times, intensities, or file names are excluded from processing to ensure data quality.

In some embodiments, the system applies a Savitzky-Golay filter to each intensity trace. The window length is adaptively chosen between 3 and 11 points based on trace length and symmetry. A polynomial order of 3 is used to preserve peak shape while suppressing high-frequency noise. This smoothing step enhances the accuracy of both apex and boundary identification without introducing artificial shoulders or peak splitting artifacts.

Apex detection is performed within a ±1-minute window centered on the explicit retention time. If no peak is detected in this primary window, a fallback mechanism expands the search range to the full chromatographic trace. The apex is identified as the local maximum within the candidate region using a robust find_peaks( ) method. If multiple peaks are found, the one with the highest intensity is retained.

In some embodiments, boundary detection is performed using a hybrid two-stage method. First, second derivative zero-crossings are computed using a numerical gradient of the smoothed intensity curve. The inflection points closest to the apex on either side are selected as initial boundaries. Second, these boundaries are extended outward until the signal drops to a “baseline threshold,” defined as 5% above the 20th percentile of the local signal intensity within a window of ±50 scans from the inflection points. This combination of geometric and statistical logic ensures reliable capture of both sharp and tailing peaks.

In some embodiments, the fraction-of-apex threshold is user-definable (default 0.05), and the maximum extension length is capped at 50 scans per side. These values were determined empirically from >10,000 manually reviewed metabolite peaks in lipidomics and polar metabolite panels. Debug-mode runs confirmed that the dual-boundary method produced <0.04 min mean error relative to expert boundaries while maintaining reproducibility across noisy signal regions.

The output is a structured CSV file containing four columns: FileName, Molecule, MinStartTime, and MaxEndTime. These boundaries represent quantification-ready peak windows that can be directly integrated into quantification tools or normalized against internal standards. Each peak passes both shape and intensity criteria and is stored in a reproducible format compatible with multi-omics integration pipelines.

In some embodiments, the software pipeline includes a set of modular components that process raw chromatographic traces to produce quantification-ready peak boundaries. The pipeline begins with a data parsing module, which reads structured CSV files containing five required fields: Times, Intensities, Molecule, Explicit RT, and File Name. The Times and Intensities fields are stored as comma-delimited strings representing one-dimensional vectors of equal length. Upon parsing, the strings are split into floating-point arrays using NumPy and validated for length consistency, numeric integrity, and the presence of non-zero intensities. Rows failing validation are excluded from downstream processing.

After parsing, a smoothing module is applied to each chromatographic trace. This module uses the scipy.signal.savgol_filter function with a default window length of 11 and a polynomial order of 3. These values are selected based on empirical trials balancing noise suppression with peak shape retention. In traces with fewer than 11 data points, the window is automatically reduced to the largest odd number below the array length. The smoothed intensity array is used for all subsequent operations, ensuring consistent baseline behavior and reducing false inflection detection.

A retention time-anchored apex detection module is used to identify the local maximum intensity value within a ±1-minute search window centered on the explicit RT value provided in the input. The apex is initially selected using the numpy.argmax( ) function applied to the search slice. If the apex intensity is less than 10 (instrument noise floor) or if no local maximum is found within the window, a fallback procedure expands the search to the entire trace using scipy.signal.find_peaks( ). Among the peaks detected in fallback mode, the candidate with the highest intensity is selected. This ensures recovery of peaks that may be shifted due to gradient variation or instrument drift.

A boundary refinement module is then invoked. First, the second derivative of the smoothed intensity trace is computed using a convolution-based method with kernel [−1, 2, −1], producing a numerical approximation of signal curvature. The two inflection points (zero-crossings) flanking the apex are identified as the initial boundaries. If zero-crossings are not found (e.g., in broad peaks or plateaus), the algorithm defaults to ±10 scans around the apex.

These initial boundaries are then extended using a percentile-based approach. A ±50-point window centered on each boundary is used to compute the 20th percentile of the intensity values, representing a dynamic estimate of the local baseline. The extension rule proceeds iteratively: from each initial boundary, the trace is extended outward in one-scan increments until the intensity falls below a threshold of 5% above the local percentile baseline, or until a maximum of 50 points has been reached. This ensures both high and low-intensity peaks are bounded appropriately.

Boundary extension logic includes a fallback to static extension if the percentile calculation fails (e.g., due to insufficient points). In such cases, the system extends ±30 points from the apex unless the trace ends earlier. If the extended boundary includes fewer than 3 total points, the peak is marked as invalid and excluded from output.

A peak validation and export module is then executed. For each valid peak, the system writes a row to a results file in CSV format with the columns: FileName, Molecule, MinStartTime, MaxEndTime, and ApexTime. These fields are computed using the actual indices of the refined boundaries and apex within the original retention time vector. All numerical values are rounded to four decimal places to ensure compatibility with downstream tools. The output file is saved using a filename that encodes the processing mode and date for auditability.

In a representative embodiment, the system was applied to a dataset of 1,000 metabolite-sample pairs extracted from C30-based lipidomics profiling of human plasma. Each molecule had a predefined explicit RT derived from pooled quality control runs. The automated pipeline was able to detect and refine boundaries for 972 out of 1,000 molecules. The average deviation between automatically computed boundaries and expert-defined ones was 0.038 minutes, and replicate peak width coefficients of variation (CVs) were under 8%. The system processed the full dataset in under 18 minutes on a 2.4 GHz quad-core workstation with 16 GB RAM.

FIG. 4 illustrates the end-to-end workflow for metabolomics peak detection. In the top panel, raw and smoothed intensity traces are shown, with the apex marked and a fallback region activated due to no peak detected in the expected ±1-minute window. The second panel shows the second derivative trace with identified zero-crossings used for initial boundaries. The third panel displays the baseline percentile region and final boundary extensions, while the last panel shows the exported MinStartTime and MaxEndTime as applied across replicate traces. This figure demonstrates the flexibility and precision of the disclosed system in adapting to complex and noisy chromatographic patterns.

### System Architecture, Workflow Integration, and Use Cases

As used herein, a “processing module” refers to an independent computational component or software subroutine that performs a defined analytical task within the pipeline, such as smoothing, peak detection, or boundary harmonization. A “dual-omics pipeline” refers to a coordinated computational workflow that concurrently processes both proteomic and metabolomic LC-MS/MS data streams to produce harmonized, quantification-ready features. A “report generation module” refers to a software component that compiles, formats, and exports peak boundary, intensity, and metadata information into structured formats for downstream interpretation or integration with laboratory information management systems (LIMS). “Interoperability” refers to the ability of different modules to exchange inputs and outputs in standardized formats, allowing for modular execution and parallelization across proteomic and metabolomic datasets. A “reference panel” refers to a curated set of peptide or metabolite features that are reproducibly detected and used as a baseline for normalization, biomarker discovery, or quality control.

In one embodiment, the system comprises four core processing modules operating in sequential workflow. The data preprocessing module accepts raw mass spectrometry files in formats including mzXML, mzML, or vendor-specific formats, extracting chromatographic traces and generating candidate peak lists. In this preprocessing embodiment, the module validates data integrity, filters incomplete traces, and creates standardized feature files containing peak candidates with associated metadata.

In some embodiments, the feature extraction module processes candidate peak data to compute analytical features required for machine learning classification. In these feature extraction embodiments, the module calculates coelution metrics by counting transitions eluting within defined time windows, computes dot product similarities by comparing observed intensity patterns to reference library spectra, determines signal-to-noise ratios by comparing peak apex intensities to local background levels, and assesses peak shape characteristics through correlation analysis with idealized peak profiles.

In another embodiment, the machine learning module implements XGBoost classification with automated hyperparameter optimization. In this ML module embodiment, the system loads training datasets containing labeled examples, performs grid search cross-validation to optimize model parameters, trains the final model on optimized parameters, and applies the trained model to score new candidate peaks. The module maintains model persistence through serialization, enabling consistent performance across analytical sessions.

In one embodiment, the postprocessing module performs peak selection, quality filtering, and result compilation. In this postprocessing embodiment, the module ranks candidates by quality scores, selects highest-scoring peaks for each analyte, applies boundary harmonization across replicates, performs retention time alignment corrections, and generates output files containing quantification-ready peak definitions with start times, end times, apex positions, and quality metrics.

The disclosed system is built on a modular, script-driven architecture with clear separation of responsibilities across processing stages. Each pipeline proteomic and metabolomic-comprises a sequence of modules organized into distinct functional directories. For example, the proteomics workflow includes directories such as /input_processing/, /model_training/, /predict_and_score/, and /boundary_align/, while the metabolomics workflow comprises /raw_data_parser/, /apex_detection/, and /boundary_extension/. Each module consumes and produces data in well-defined CSV or tab-delimited formats, allowing independent execution or scheduled batch runs on high-performance clusters. Inputs to the system include Skyline or mProphet reports for proteomics and raw trace CSVs for metabolomics, while outputs are standardized boundary files, classifier confidence tables, and summary reports.

In some embodiments, a workflow integration controller coordinates proteomic and metabolomic processing by tracking common metadata such as sample IDs, injection order, and batch identifiers. This controller is implemented as a Python orchestration layer that maintains a global index of sample files and invokes appropriate module sequences based on data type. For dual-omics datasets, shared sample identifiers are used to link proteomic and metabolomic boundaries and store them under a unified namespace. Configuration files (e.g., config.yaml) define user-specific parameters such as classifier thresholds, smoothing window sizes, fallback rules, and output filenames. Each run is automatically logged with timestamps and runtime statistics for reproducibility.

In some embodiments, the system is applied in clinical and translational biomarker discovery workflows. In a typical proteomics use case, DIA-PASEF data are collected from patient cohorts with well-defined clinical phenotypes. Using the labeling and prediction modules, the system scores and filters over 150,000 candidate PSMs per sample, retaining only high-confidence peaks with reproducible apex and boundary definitions. The resulting quantification-ready peptide list is used to generate MRM transition panels, which are deployed in targeted quantification studies to validate candidate biomarkers across larger cohorts. All steps—from classifier scoring to boundary harmonization—are performed automatically, reducing panel development time from multiple weeks to under two hours per dataset.

In a separate use case, the system supports high-throughput metabolomics-based quantification for population-scale studies or pharmaceutical trials. Full-scan Orbitrap data acquired across thousands of samples are processed in batch mode to generate harmonized boundary files for targeted and untargeted metabolites. The system applies fallback logic, dynamic smoothing, and percentile-based boundary extension to maximize recall of low-abundance or drifted features. Outputs are merged with patient metadata and prepared for pathway enrichment analysis, exposure-response modeling, or machine learning-based classification. This use case enables scalable and reproducible metabolic phenotyping across diverse sample types and acquisition platforms.

In some embodiments, the system is deployed in routine quality control pipelines within pharmaceutical and biotechnology manufacturing settings. Standardized peptide or metabolite panels are used as batch-level QC markers. The system automatically detects peaks, applies classifier thresholds, and flags any deviations in apex RT, peak width, or signal-to-noise ratio beyond user-specified tolerances. The report generation module compiles QC flags, deviation plots, and pass/fail summaries, which are exported in compliance-ready formats. This enables fully automated, reproducible tracking of batch integrity and LC-MS performance over time without requiring manual review.

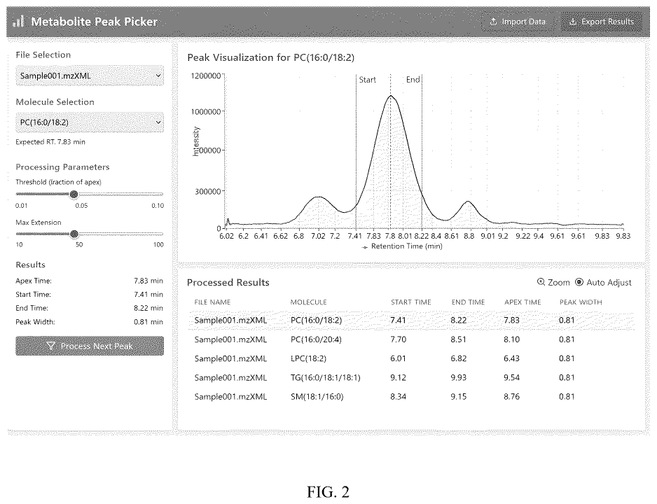

In some embodiments, the system includes an interactive graphical user interface (GUI) for manual inspection and quality control of peak detection results, particularly in the metabolomics workflow. As shown in FIG. 2, users can select sample files and target metabolites from dropdown menus, visualize smoothed intensity traces, and inspect assigned peak boundaries and apex positions. The GUI displays baseline percentile thresholds and signal cutoff markers, allowing users to adjust parameters such as “percent of apex” or “max extension length” and view the real-time impact on peak definition. It also enables toggling between automatic and manual modes, supporting exploratory data review during method development or exception handling in QC pipelines.

In some embodiments, the system supports cross-platform interoperability via standardized output schemas. All modules export results in .csv or .tsv formats with column headers defined in the system schema file. File formats are intentionally aligned across proteomic and metabolomic pipelines, so downstream users can perform dual-omics integration, normalization, or visualization. For example, a unified complete_peaking_output.csv may be created containing both peptide and metabolite entries under common sample IDs, enabling joint pathway enrichment or machine learning classification. The system can be deployed locally or containerized using Docker, Singularity, or Conda-based environments. This supports reproducibility, easy scaling to cloud platforms (e.g., AWS Batch or Google Cloud Run), and compatibility with institutional high-performance computing (HPC) clusters.

In some embodiments, a report generation module compiles peak-level results across runs and formats them into Excel-compatible or JSON summary reports. Each report includes a header with date, runtime, input parameters, software version, and number of processed samples. Optional LIMS-compatible exports are provided using sample barcodes and standardized filenames (e.g., CP_Export_<sampleID>_<date>.csv). The report generator may also annotate failed or borderline peaks based on classifier score thresholds or missing baseline coverage. When used in biomarker discovery workflows, the exported database entries include for each feature a unique identifier, apex retention time, peak width, quality score, and diagnostic metadata-such as inclusion status in machine learning-derived panels or prior disease association scores-enabling downstream validation or decision support integration. This enables both automated and manual review workflows in regulated or research environments.

### Multi-Omics Data Processing Pipeline

The invention encompasses a comprehensive pipeline for processing proteomics, metabolomics, and lipidomics data from biological samples, particularly blood and plasma specimens. The system begins with data preprocessing to generate feature files containing candidate peaks for target analytes across all molecular classes. For proteomics data, this involves generating mProphet feature files using mass spectrometry software tools that analyze Selected Reaction Monitoring (SRM) or Multiple Reaction Monitoring (MRM) datasets. The preprocessing module creates comprehensive lists of candidate peaks that could potentially be selected for each analytical fraction, establishing the foundation for subsequent machine learning analysis.

Feature extraction encompasses multiple analytical approaches tailored to different molecular classes. For proteomics applications, key features include coelution count calculated by identifying transitions with apex retention times falling within ±0.05 to ±0.2 minute windows and incrementing counters for each qualifying transition, dot product scores computed by normalizing observed and reference intensity vectors to unit length and calculating their cosine similarity with values ranging from 0 to 1, signal-to-noise ratios determined by dividing peak apex intensity by the standard deviation of baseline noise calculated from flanking chromatographic regions, shape correlation coefficients computed using Pearson correlation between observed peak profiles and idealized Gaussian or exponentially modified Gaussian peak shapes with correlation values exceeding 0.7 indicating acceptable peak quality, and raw peak intensities measured as integrated areas under chromatographic curves within defined retention time boundaries. In these feature extraction embodiments, coelution count serves as the most predictive feature for peak quality assessment, as simultaneous elution of multiple transitions provides strong evidence for authentic analyte detection rather than random noise or interference signals.

The system incorporates specialized algorithms for metabolomics data processing that differ significantly from proteomics approaches. Metabolomics peak detection utilizes a second derivative method beginning with intensity signal smoothing using Savitzky-Golay filtering with optimized parameters including window length of 11 data points and polynomial order of 3. The algorithm computes second derivatives of smoothed intensity profiles to identify changes in concavity marking peak boundaries. Zero-crossings of the second derivative define transition points from concave up to concave down regions, establishing start and end points for each metabolite peak. Background correction involves calculating median intensity thresholds outside peak regions, with iterative boundary adjustments ensuring peaks do not extend into noise areas. Width correction removes outliers using interquartile range (IQR) methods, followed by standardization based on samples with largest total peak areas. Peak boundaries are adjusted using extension factors to account for asymmetry commonly observed in metabolomics chromatography.

### Blood Matrix Pattern Recognition

A critical innovation involves recognizing and utilizing consistent noise patterns inherent in blood and plasma samples. Analysis of thousands of plasma specimens reveals reproducible interference patterns originating from high-abundance proteins, lipids, and other prevalent blood matrix components. These patterns, while traditionally considered detrimental to analytical depth, provide valuable landmarks for automated peak identification when properly characterized. The consistent reproducibility of these matrix-specific signals across different specimens enables precise peak-picking without requiring exogenous internal standards, significantly reducing analytical costs and complexity.

The system catalogues common blood matrix interference signatures including specific retention time windows, intensity profiles, and co-elution patterns associated with abundant plasma proteins such as albumin, immunoglobulins, and apolipoproteins. Machine learning algorithms incorporate these pattern recognition capabilities to distinguish between genuine analyte signals and matrix interferences, while simultaneously using matrix patterns as reproducible reference points for retention time alignment and peak quality assessment. This approach transforms traditional analytical challenges into systematic advantages for automated data processing.

### Machine Learning Model Training and Optimization

The core machine learning framework utilizes XGBoost gradient boosting algorithms optimized through comprehensive hyperparameter tuning. Key parameters include maximum tree depth controlling model complexity and pattern recognition capability, learning rates determining convergence speed and stability, gamma values providing regularization to prevent overfitting, lambda regularization terms for L2 penalty application, and scale positive weight parameters for handling imbalanced datasets common in clinical samples. Cross-validation employs grid search methodologies to optimize these parameters across diverse training datasets.

Training datasets incorporate manually curated data from clinical specimens including samples from patients with cancer, cardiovascular disease, neurodegenerative disorders, and autoimmune conditions, alongside matched control specimens. This clinical training approach enables the model to recognize disease-specific analytical patterns and background variations characteristic of different pathological states. The diversity of training data ensures robust performance across varied clinical applications while maintaining sensitivity to subtle biomarker signals that may be disease-specific.

Model validation employs multiple approaches including technical replicates, independent validation cohorts, and cross-platform comparisons. Performance metrics focus on peak identification accuracy, false discovery rates, and quantitative reproducibility across different analytical conditions. The trained models demonstrate superior performance compared to conventional peak picking algorithms, particularly for low-abundance analytes in complex biological matrices.

### Model Prediction and Scoring Process

In one embodiment, the trained machine learning model applies predictive analysis to new datasets by processing feature matrices extracted from candidate peaks. In this prediction embodiment, the trained XGBoost model receives input vectors containing coelution count, dot product score, signal-to-noise ratio, shape correlation, and peak intensity values for each candidate peak. The model outputs probability scores ranging from 0 to 1, where values approaching 1 indicate high-quality peaks and values approaching 0 indicate low-quality or noise peaks.

In some embodiments, the prediction process involves loading the serialized XGBoost model from persistent storage and applying it to feature tables containing thousands of candidate peaks per sample. In these embodiments, the model processes features in batch mode, generating quality scores for all candidates simultaneously. The scoring algorithm applies the decision tree ensemble to each feature vector, combining individual tree predictions through weighted voting to produce final quality scores.

In another embodiment, quality score thresholds are applied during the prediction phase to filter candidates. In this threshold embodiment, peaks scoring below 0.5 are automatically excluded from further consideration, while peaks scoring above 0.8 are marked as high-confidence selections. Peaks scoring between 0.5-0.8 undergo additional validation steps including retention time consistency checks and replicate correlation analysis.

In one embodiment, postprocessing involves selecting the highest-scoring prediction for each target analyte from multiple candidate peaks. In this selection embodiment, the system groups all candidate peaks by analyte identifier and ranks them by machine learning quality scores. For each analyte, the candidate with the highest quality score is retained as the representative peak for quantification. In some embodiments, tie-breaking procedures address cases where multiple candidates receive identical or near-identical scores, applying secondary criteria including peak intensity, retention time proximity to expected values, and co-elution count sequentially until a single peak is selected.

### Multi-Point Normalized Protein Expression (MNPX) System

The invention incorporates an advanced normalization methodology termed Multi-point Normalized Protein expression (MNPX) that addresses analytical variations across different detection clusters. Recognition that proteins, metabolites, and lipids exhibit different analytical behaviors based on physicochemical properties necessitates cluster-specific normalization approaches. The system groups analytes into clusters based on peak intensities, hydrophobicities, retention time behaviors, and other analytical characteristics.

Each analytical cluster employs tailored normalization strategies with multiple reference points selected based on cluster-specific criteria. For high-abundance protein clusters, normalization may utilize median intensity values of stable housekeeping proteins, while low-abundance protein clusters employ different reference standards optimized for sensitivity. Metabolomics clusters incorporate pathway-specific normalization approaches accounting for metabolic flux variations, and lipid clusters utilize class-specific normalization addressing lipid extraction and ionization efficiency differences.

The MNPX system enables direct comparison of analyte abundances across different samples and analytical conditions, facilitating identification of disease-associated molecular changes. This multi-reference approach provides superior quantitative accuracy compared to single-point normalization methods commonly used in proteomics and metabolomics applications.

### Quality Control and Validation Parameters

The system implements comprehensive quality control measures ensuring reliable peak identification across diverse analytical conditions. Signal quality parameters include minimum signal-to-noise ratio requirements of 1.5 or greater, retention time consistency requirements exceeding 1.5 minutes to avoid early-eluting interferences, and background interference controls requiring background signals at least one order of magnitude lower than analyte peak intensities. These thresholds were established through analysis of thousands of clinical samples across multiple analytical platforms.

Additional validation criteria include fragment ion ratio consistency within ±20-30% of reference values, ensuring fragmentation pattern stability across different analytical runs. Spectral similarity assessments require dot product or correlation scores exceeding 0.5-0.6 when comparing observed fragmentation patterns to reference library spectra. Co-elution validation ensures transition apexes align within ±0.05-0.2 minute windows, confirming consistent elution profiles for authentic analyte signals.

Peak shape assessments utilize correlation analyses comparing individual peak profiles to median shapes calculated across all transitions for each analyte. These multi-parameter quality assessments provide robust discrimination between authentic analyte signals and analytical artifacts, ensuring high confidence in quantitative results used for clinical decision-making.

### Clinical Diagnostic Applications

The integrated platform enables simultaneous analysis of proteins, metabolites, and lipids from single biological samples, providing comprehensive molecular characterization for disease diagnosis and monitoring. Disease-specific molecular signatures are identified through comparison of patient samples to reference patterns established from healthy control populations and disease-specific cohorts. The system processes molecular signatures associated with cancer types including breast, colorectal, lung, ovarian, pancreatic, and prostate cancers, cardiovascular diseases, neurodegenerative conditions including Alzheimer's disease, and autoimmune disorders.

Diagnostic workflows involve obtaining blood or plasma samples from subjects, processing samples through the automated peak picking pipeline to identify molecular signatures comprising proteins, metabolites, and lipids, comparing identified signatures to disease-specific reference patterns stored in comprehensive databases, and determining disease status based on signature comparison algorithms. The multi-omics approach provides enhanced diagnostic accuracy compared to single-analyte class approaches, leveraging complementary information from different molecular layers to improve clinical decision-making.

The system supports both screening applications for asymptomatic populations and diagnostic confirmation for symptomatic patients. Integration with clinical laboratory information systems enables automated reporting of results with appropriate clinical context and recommendations for follow-up testing or therapeutic interventions based on observed molecular patterns.

### Implementation and Scalability

The pipeline architecture supports high-throughput clinical applications through automated processing workflows and scalable computing infrastructure. Data processing modules operate independently, enabling parallel processing of multiple samples and analytical batches. The system accommodates different mass spectrometry platforms and analytical configurations through configurable parameters and platform-specific optimization protocols.

Quality assurance includes automated data integrity checks, analytical performance monitoring, and systematic validation protocols ensuring consistent performance across different laboratory environments. The modular design enables customization for specific clinical applications while maintaining core analytical capabilities across diverse implementation scenarios.

Integration with laboratory information management systems (LIMS) provides seamless data flow from sample collection through result reporting, supporting regulatory compliance requirements for clinical diagnostic applications. The system maintains detailed audit trails and analytical metadata supporting quality assurance and regulatory submission requirements for clinical implementation.

### Model Prediction and Scoring Process

In one embodiment, the trained machine learning model applies predictive analysis to new datasets by processing feature matrices extracted from candidate peaks. In this prediction embodiment, the trained XGBoost model receives input vectors containing coelution count, dot product score, signal-to-noise ratio, shape correlation, and peak intensity values for each candidate peak. The model outputs probability scores ranging from 0 to 1, where values approaching 1 indicate high-quality peaks and values approaching 0 indicate low-quality or noise peaks.

In some embodiments, the prediction process involves loading the serialized XGBoost model from persistent storage and applying it to feature tables containing thousands of candidate peaks per sample. In these embodiments, the model processes features in batch mode, generating quality scores for all candidates simultaneously. The scoring algorithm applies the decision tree ensemble to each feature vector, combining individual tree predictions through weighted voting to produce final quality scores.

In another embodiment, quality score thresholds are applied during the prediction phase to filter candidates. In this threshold embodiment, peaks scoring below 0.5 are automatically excluded from further consideration, while peaks scoring above 0.8 are marked as high-confidence selections. Peaks scoring between 0.5-0.8 undergo additional validation steps including retention time consistency checks and replicate correlation analysis.

### Peak Selection and Postprocessing

In one embodiment, postprocessing involves selecting the highest-scoring prediction for each target analyte from multiple candidate peaks. In this selection embodiment, the system groups all candidate peaks by analyte identifier and ranks them by machine learning quality scores. For each analyte, the candidate with the highest quality score is retained as the representative peak for quantification.

In some embodiments, tie-breaking procedures address cases where multiple candidates receive identical or near-identical scores. In these tie-breaking embodiments, secondary criteria include peak intensity (higher preferred), retention time proximity to expected values (closer preferred), and co-elution count (higher preferred). These criteria are applied sequentially until a single peak is selected for each analyte.

In another embodiment, postprocessing includes boundary harmonization across technical replicates and sample batches. In this harmonization embodiment, the system identifies the most intense peak instance for each analyte across all files and propagates its start and end boundaries to all other instances of the same analyte. This ensures consistent integration windows across replicates, reducing quantitative variability.

In some embodiments, retention time alignment is performed during postprocessing by computing average boundary offsets relative to peak apex positions. In these alignment embodiments, global average left and right offsets are calculated from the highest-quality instances and applied to all samples, compensating for chromatographic drift while maintaining peak shape characteristics.

### System Architecture and Module Implementation

In one embodiment, the system comprises four core processing modules operating in sequential workflow. The data preprocessing module accepts raw mass spectrometry files in formats including mzXML, mzML, or vendor-specific formats, extracting chromatographic traces and generating candidate peak lists. In this preprocessing embodiment, the module validates data integrity, filters incomplete traces, and creates standardized feature files containing peak candidates with associated metadata.

In some embodiments, the feature extraction module processes candidate peak data to compute analytical features required for machine learning classification. In these feature extraction embodiments, the module calculates coelution metrics by counting transitions eluting within defined time windows, computes dot product similarities by comparing observed intensity patterns to reference library spectra, determines signal-to-noise ratios by comparing peak apex intensities to local background levels, and assesses peak shape characteristics through correlation analysis with idealized peak profiles.

In another embodiment, the machine learning module implements XGBoost classification with automated hyperparameter optimization. In this ML module embodiment, the system loads training datasets containing labeled examples, performs grid search cross-validation to optimize model parameters, trains the final model on optimized parameters, and applies the trained model to score new candidate peaks. The module maintains model persistence through serialization, enabling consistent performance across analytical sessions.

In one embodiment, the postprocessing module performs peak selection, quality filtering, and result compilation. In this postprocessing embodiment, the module ranks candidates by quality scores, selects highest-scoring peaks for each analyte, applies boundary harmonization across replicates, performs retention time alignment corrections, and generates output files containing quantification-ready peak definitions with start times, end times, apex positions, and quality metrics.

### Enhanced Quality Control Implementation

In one embodiment, quality control parameters are systematically applied during multiple processing stages to ensure analytical reliability. In this quality control embodiment, signal-to-noise ratio requirements of 1.5 or greater are enforced by comparing peak apex intensities to local baseline levels calculated from surrounding chromatographic regions. Peaks failing this criterion are automatically excluded from candidate lists.

In some embodiments, retention time consistency validation requires peak apex positions to fall within acceptable windows around expected retention times. In these retention time embodiments, acceptable windows typically span ±0.5 minutes from reference values established through quality control sample analysis. Peaks eluting outside these windows undergo additional scrutiny including spectral similarity verification and co-elution pattern confirmation.

In another embodiment, background interference assessment ensures peak signals exceed local background by at least one order of magnitude. In this background control embodiment, local baseline levels are calculated using median intensities from chromatographic regions flanking each peak. Peak intensities must exceed 10× the local baseline to pass quality criteria, ensuring quantitative reliability in complex biological matrices.

In some embodiments, fragment ion ratio validation confirms fragmentation pattern consistency across analytical runs. In these fragmentation embodiments, observed ion ratios are compared to reference values established during method development, with acceptable deviation ranges of ±20-30% from reference patterns. Peaks exhibiting ion ratios outside these ranges are flagged for manual review or automatic exclusion.

### Step-by-Step Diagnostic Methodology

In one embodiment, the diagnostic workflow comprises systematic steps for disease detection using multi-omics molecular signatures. The first step involves obtaining biological samples, typically blood or plasma specimens, from subjects using standard clinical collection procedures including proper anticoagulation, centrifugation, and storage protocols to maintain molecular stability.

In this diagnostic embodiment, the second step processes samples through the automated peak picking pipeline to identify and quantify molecular signatures. This processing includes sample preparation using chemical-biological depletion methods for plasma proteins, metabolite extraction using modified MTBE/MeOH/H2O protocols, and lipid extraction optimized for comprehensive coverage. Mass spectrometry analysis employs targeted methods including dynamic Selected Reaction Monitoring (dSRM) for proteins and dynamic Multiple Reaction Monitoring (dMRM) for metabolites and lipids.

In some embodiments, the third diagnostic step involves comparing identified molecular signatures to disease-specific reference patterns stored in comprehensive databases. In these comparison embodiments, patient signatures comprising proteins, metabolites, and lipids are matched against reference panels established from validated disease cohorts and healthy control populations. Statistical comparison methods include t-tests, fold-change analysis, and machine learning classification algorithms trained on clinical datasets.

In another embodiment, the fourth diagnostic step determines disease status through signature pattern analysis. In this determination embodiment, classification algorithms process the top 1000 most discriminatory features, typically comprising approximately 794 proteins, 90 metabolites, and 117 lipids per disease panel. Disease likelihood scores are calculated based on signature similarity to reference patterns, with threshold values optimized for clinical sensitivity and specificity requirements.

In some embodiments, diagnostic results include confidence metrics, differential diagnosis considerations, and recommendations for confirmatory testing. In these reporting embodiments, results are formatted for clinical laboratory information systems with appropriate interpretive guidance, quality control flags, and follow-up recommendations based on observed molecular patterns and clinical context.

### EXPERIMENTAL VALIDATION AND EXAMPLES

### Example 1—Proteomics Classifier Performance and Boundary Harmonization

In one example, the proteomics pipeline was validated using DIA mass spectrometry files acquired from human plasma samples on the timsTOF HT platform. Each file generated more than 150,000 peptide-spectrum matches (PSMs), which were processed using DIA-NN and proteotypic high intensity peptides were validated using Skyline. A ground-truth training dataset containing 37,646 peptide transition instances was created, as shown in ‘train_dataset_sample.csv’. Each instance included a binary label (‘Label’, 0 or 1) assigned by expert curation following the protocol as shown in a training dataset of 37,646 labeled transitions compiled from curated Skyline outputs. Candidate peaks were evaluated based on retention time alignment, dot-product score to library spectra, signal-to-noise ratio, co-elution of fragment ions, and peak shape symmetry.

The dataset included key feature columns such as ‘main_var_Intensity’, ‘var_Library_intensity_dot-product’, ‘var_Shape_(weighted)’, ‘var_Co-elution_(weighted)’, ‘var_Co-elution_count’, and ‘var_Signal_to_noise’, which were extracted from Skyline or mProphet outputs and computed using post-processing scripts. These features were used to train an XGBoost classifier with model hyperparameters tuned using five-fold cross-validation to maximize AUROC, which averaged 0.96 across all folds. The classifier was serialized and integrated into the prediction module for downstream scoring.

Manually evaluators curated the training data following the labeling protocol, yielding over 3,000 annotated peaks across a variety of conditions and technical replicates. Each peak was evaluated for reproducibility across files, dot-product similarity to library spectra, and co-elution of fragment ions. These labels were used to train an XGBoost classifier with features including intensity variance, symmetry of the elution profile, dot-product similarity, and co-elution count. Model hyperparameters were optimized using five-fold cross-validation to achieve an average AUROC of 0.96.

### Example 2: Metabolomics Peak Detection Across Multiple Chromatographic Modes

In another embodiment, the metabolomics pipeline was validated using liquid chromatography-mass spectrometry (LC-MS/MS) datasets obtained from multiple chromatographic separation modes, including C30-based lipid profiling, C18-based reverse-phase separation, and hydrophilic interaction chromatography (HILIC) for polar metabolite analysis. The datasets collectively included over 3,000 metabolite-sample pairs, acquired using an Orbitrap-class mass spectrometer configured with optimized gradient conditions tailored to each chromatographic mode. For instance, the C30 column was used to enhance retention of lipophilic species such as triglycerides and sphingolipids, the C18 column targeted mid-polar analytes, and the HILIC column enabled detection of polar metabolites including amino acids and nucleosides.

Each data file consisted of trace-level intensity arrays indexed by retention time, along with metadata such as molecule identifier, quality control-derived reference retention times, and run identifiers. A signal processing module parsed each chromatographic trace, applying a smoothing algorithm to reduce noise. Apex detection was performed by searching within a specified retention time window around the expected elution point; if no peak met the threshold criteria, a fallback expanded the search across the full trace. Following apex identification, a two-stage boundary detection algorithm was applied. First, the curvature of the smoothed trace was analyzed to locate inflection points, establishing initial boundaries. Then, these were adaptively extended until the signal intensity fell below a dynamic threshold, computed using local percentile values. In cases with insufficient surrounding data, static fallback rules were applied, and total extension per side was capped to avoid overestimation.

Across the datasets, the algorithm identified 972 high-confidence peaks in the C30 lipidomics dataset, 1,083 in the C18 dataset, and 1,026 in the HILIC dataset. Comparison with manually annotated peaks showed a mean deviation of 0.038 minutes for boundary placement. Peak width coefficients of variation (CVs) across replicate runs were under 8%, and average processing time per dataset was under 20 minutes using a standard quad-core processor with 16 GB of memory. These results demonstrate the reproducibility and scalability of the system across varied chromatographic strategies.

### Example 3: Dual-Omics Panel Generation for Clinical Discovery

In a third example, a dual-omics use case was tested using paired proteomic and metabolomic data from 50 patient samples. Proteomic data were processed using the same classifier and harmonization pipeline as described above, resulting in 1,550 peptide features with high confidence scores. Metabolomics traces were acquired from the same samples using HILIC, C18, and C30 columns, processed using the boundary detection algorithm described above. Of 243 metabolite features, 102 were retained after quality filtering. Sample IDs and metadata were mapped to generate a unified dataset.

The final dual-omics reference panel included 57 peptides and 102 metabolites with reproducible retention time boundaries and classifier scores. Each entry included apex time, start and end boundaries, and a quality score. Outputs were merged into a single annotated table with sample ID, feature ID, feature type (peptide or metabolite), and diagnostic panel inclusion. Cross-validation of disease status using this panel yielded 89% accuracy. The results were exported in a format suitable for downstream integration with statistical and machine learning pipelines.

### Example 4: Automated Quality Control in Batch Monitoring

In a fourth example, the system was validated in a pharmaceutical quality control setting. A reference mixture of twelve peptides and fifteen metabolite internal standards was used to monitor batch reproducibility. Samples were acquired over 30 production batches across three LC-MS instruments using a combination of HILIC and C18 columns for internal standard coverage across polar and non-polar compounds. The automated pipeline applied smoothing, peak scoring, and harmonization as previously described. For each feature, apex deviation and width ratio were computed relative to batch baselines. Deviation thresholds included ±0.2 minutes for retention time and 1.5× for peak width.

Results were exported to standardized CSV and XML formats for upload to the laboratory information management system (LIMS). Reports contained sample ID, compound ID, deviation metrics, and pass/fail status. Of 30 batches, three were flagged for borderline deviations (>0.22 minutes), later attributed to pressure instability. No false positives were observed. All expected peaks were detected, confirming the system's utility in GMP-grade automated quality assurance.

These examples confirm that the disclosed system provides automated, reproducible, and scalable peak detection and refinement for both proteomic and metabolomic LC-MS/MS workflows. The modular design supports dual-omics integration and quality control applications, enabling consistent peak calling without manual review and minimizing boundary variability across samples, batches, and instruments.

### Example 5: Complete360: A Multi-Omics Platform for the Targeted Molecular Characterization of Plasma from Different Diseases

Blood is one of the most accessible clinical samples for liquid biopsy, making it invaluable for molecular diagnostics in human disease. Several methods have been previously reported for detecting and quantifying disease associated molecular changes in blood, particularly nucleic acids in the context of human cancers, and some of these methods have successfully led to commercial applications in clinical settings¹⁻³. Some disease development such as certain cancers is often associated with genetic alterations such as mutations and translocations, these changes can be measured from circulating DNA in the blood⁴. However, many human diseases—such as cardiovascular, neurodegenerative, and autoimmune disorders—do not involve genomic alterations and cannot be assessed through sequencing methods. Additionally, genetic predispositions to cancers, once measured in a person to assess cancer risks, the subsequent measurement provides no additional information for clinical intervention, which limits the clinical utilities for sequencing technologies aimed at detecting nucleic acid changes in disease management.

In contrast, proteins and small molecules (metabolites and lipids) in human blood serve as excellent surrogates that reflect real-time changes in health and disease status^{5, 6}. Virtually if all human diseases could be monitored through variations in protein or small molecule surrogate levels in blood, most clinical tests can be achieved by measuring proteins or small molecules⁷. However, accurately measuring these molecules in blood has proven to be a significant challenge, limiting their clinical applications.

Traditional methods for measuring proteins and metabolites in blood can be categorized into two main approaches. The first is the affinity-based method. The second approach involves mass spectrometry, which can be performed in either targeted or untargeted modes. The advent of advanced mass spectrometers, such as the timsTOF HT from Bruker and Astral from ThermoFisher, in combination with low abundant blood protein enrichment strategies, has allowed researchers to detect ˜6,000 proteins from plasma samples using untargeted mass spectrometry^{8, 9}. While untargeted mass spectrometry provides a broader range of identified proteins or small molecules, it generally lacks sensitivity and/or specificity. Using targeted proteomics, Applicants achieved highly sensitive detection of specific mutated forms of proteins as early-stage cancer-specific protein changes, and extremely low abundant neoantigens through modified targeted approaches, demonstrating high sensitivity after a series of parameter optimizations aimed at ultra-low abundance detection^{6, 10-13}.

Applicants hypothesized that if they could recursively optimize the detection for each protein individually and compile these methods into a single assay, Applicants could ensure accurate detection and quantification across all human blood proteins, achieving the sensitivity and reproducibility required for clinical applications. By optimizing liquid chromatography-mass spectrometry (LC-MS) parameters and developing customized sample preparation procedures for each of all specific protein targets, Applicants reached unprecedented sensitivity for each protein and small molecule target. Here, Applicants describe the development of Complete360, a multi-omics platform designed to advance both research and clinical investigations, capable of detecting over 10,000 human proteins and more than 2,000 metabolites and lipids in plasma samples. This platform operates through a fully optimized and automated process that integrates sample preparation, mass spectrometry method clusters, and data analysis. Complete360 addresses the limitations of traditional mass spectrometry by significantly enhancing the reproducibility and detectability of proteins, metabolites, and lipids often missed by other technologies. It provides more accurate multi-omics data by capturing higher magnitudes of biological differences between diseased bloods and controls. This practicality ensures its feasibility for real-world clinical applications.

### Results

### Complete360 Platform: Integrating Extensive Data Acquisition and Curation Using Artificial Intelligence (AI)

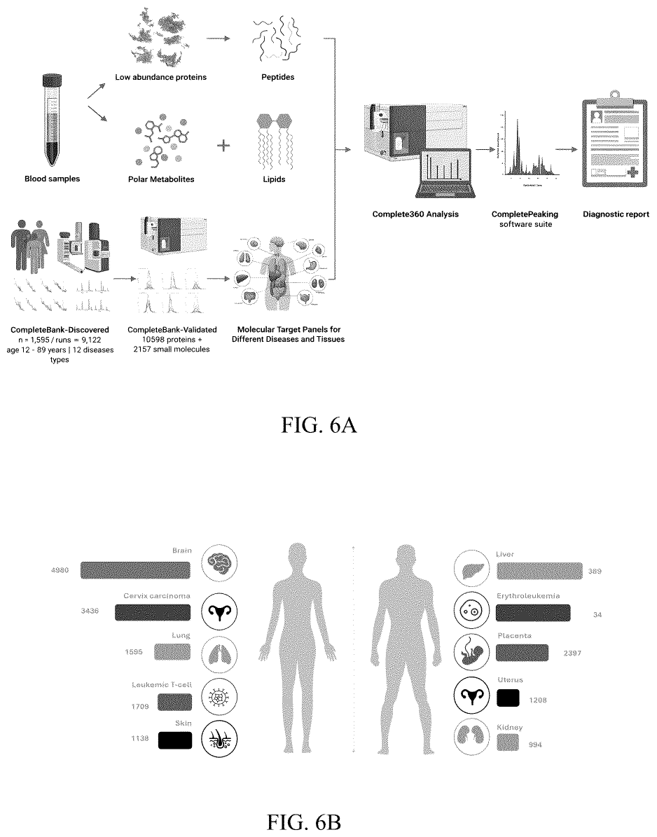

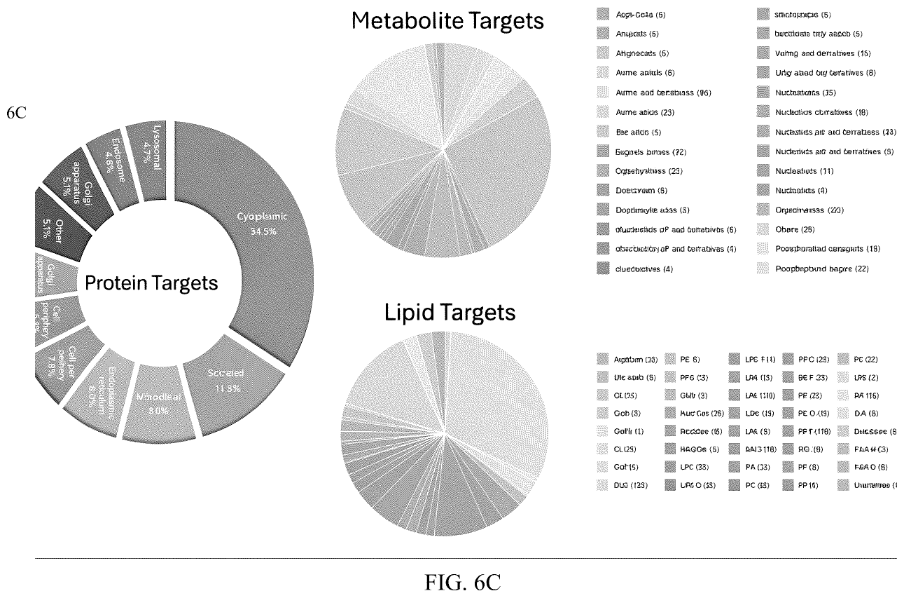

The Complete360 platform comprises of 1) method to extract plasma proteins, metabolites, and lipids from blood samples and mass spectrometry analysis, 2) CompleteBank for database construction from multi-omics analyses of blood samples from healthy and different disease individuals, 3) multi-omics targeted assay development, and 4) data analysis using CompletePeaking Algorithm for report (FIG. 6A).

Method for extraction of plasma proteins, metabolites, and lipid: Applicants first established a method and standard operating procedure (SOP) to extract plasma proteins, metabolites, and lipids. The extraction method was optimized to increase the sensitivity and reproducibility for protein, metabolite, and lipid detection. The proteomic method is based on the depletion of high abundant proteins using a chemical depletion method, after depletion, the proteins were digested to peptides and fractionated 96 fractions using offline bRPLC. Each fraction was analyzed by LC-MS/MS analysis using RPLC for online peptide separation and MS/MS using timsTOF HT. The details of the method is described in the method session. The metabolomic analysis is based on the hydrophilic extraction of metabolites using methanal and metabolites were analyzed by LC-MS/MS using online HILIC separation and mass spectrometric analyses using QE HFX in positive and negative modes. The lipidomic method includes the extraction of lipids from blood samples using a modified MTBE/MeOH/H2O extraction method and analyzed by LC-MS/MS using C18 column coupled with QE HFX in both positive and negative molds.

Database construction for CompleteBank: Applicants then applied the established method and SOPs to the analysis of various human samples and constructed a comprehensive database, termed CompleteBank. In the first phase, Applicants created a discovery database encompassing 17,328 proteins and 2,927 metabolites/lipids observed in various human samples using data-dependent acquisition (DDA) LC-MS/MS methods from different human samples. This module, termed CompleteBank-Discovered (FIG. 6A), serves as a repository for preliminary detection parameters of blood signatures, laying the groundwork for targeted assay development. To assess the depth and uniqueness of CompleteBank-Discovered, Applicants compared the protein list with the recently published Human Proteome Project (HPP) database¹⁴. Remarkably, Applicants identified 536 proteins from the PE2-5 groups, marking the first-ever detection of these proteins by mass spectrometry, representing approximately 2.74% of the human proteome. This achievement is particularly significant given that these proteins were detected in blood, where protein identification is inherently more challenging. The findings not only expand the known repertoire of detectable human proteins but also highlight the completeness and robustness of the foundational database.

Multiplex targeted assay development: The targeted assays were developed to ensure the detectability and reliability of the proteins, metabolites, and lipids identified in the CompleteBank-Discovered. This involved the optimization for each target molecule on liquid chromatographic separation, mass spectrometric analysis, and transition selection, aimed at maximizing detection sensitivity and specificity. Through this process, Applicants curated targeted mass spectrometric assays using dynamic Selected Reaction Monitoring (dSRM) corresponding to 10,598 blood proteins and 2,157 small molecules, which collectively form CompleteBank-Validated module (FIG. 6A). This module has been developed on triple quadrupole (QqQ) mass spectrometers. Over 600,000 QqQ raw spectrum files (publicly accessible through ProteomeXchange in the PeptideAtlas SRM Experiment Library (PASSEL) (identifier PASS05916) were manually reviewed to ensure data quality. The CompleteBank-Validated module is enriched with precise detection parameters, including optimization on retention times, m/z values for MS1 and MS2 fragments, collision energies, source parameters and blood-specific noises as well as target-specific noises. Together, with the established SOPs for peptide or small molecular extraction, and protection peptides used for each target through the proprietary MaxRec technology¹².

To evaluate the sources from the optimized CompleteBank-Validated proteins, the UniProt tissue annotation database (UP_TISSUE) was used, and Applicants found that the validated blood proteins developed in the Complete360 platform came from all major human tissues or organs (FIG. 6B). Additionally, subcellular localization analysis revealed the following distribution: 34.5% cytoplasmic, 11.8% secreted, 9% cytoskeletal, 8.2% mitochondrial, 8.0% endoplasmic reticulum, 7.8% cell projection, 6.5% Golgi apparatus, 4.7% cytoplasmic vesicle, and 4.4% endosomal proteins, etc (FIG. 6C); and 2,157 small molecules, where there are 762 polar metabolites from different classes (organic acids, amino acids, nucleotides, carbohydrates, etc.), covering most of the human metabolic, drug and disease pathways, and 1,395 lipid species across more than 24 (sub)classes (including AcylCarn, Bile acids, CE, CL, DAG, DE, DG, FFA, HexCer, LPC, LPE, LPG, LPI, MAG, PA, PC, PE, PG, PI, PL, PS, PIP, SIP, SM, SP, SPN, TAG, TG)¹⁵⁻¹⁹ (FIG. 6C).

Data analysis using CompletePeaking Algorithm: During the method development phase, Applicants extensively relied on manual curation, which proved indispensable for enhancing the quality and reliability of Applicants' database. This meticulous process allowed us to fine-tune detection parameters. Over the course of approximately six years, Applicants iteratively optimized these parameters while documenting more than 9,000 blood samples. This process enabled high sensitivity for each target detected via mass spectrometry. With these large manually curated datasets in hand, Applicants developed an AI-based learning and peak-picking system, which Applicants named CompletePeaking (FIG. 6A). This system performs data analysis for each sample using comprehensive pattern-recognition algorithms to improve the identification of precise peaks for each target, with particular emphasis on determining the optimal transition(s)²⁰. The manually curated spectral files served as the training set for the CompletePeaking algorithm. With CompletePeaking, the detection of all analytes has been consolidated into a suite of mass spectrometry assay clusters designed to maximize both sensitivity and reproducibility while minimizing runtime. The development of these assays incorporated several key considerations, including separation of high- and low-abundance molecules, resolution of co-eluted targets, ensure retention time reproducibility, management of co-eluting noise analytes, and minimizing Run Time, which detailed in method session.

Worthy of highlighting, as previously mentioned in management of co-eluting noise analytes, when analyzing blood samples, Applicants consistently observed similar noise patterns across different specimens. These patterns likely originate from common high-abundance proteins or blood matrix components, such as lipids and other prevalent substances^{21, 22}. These elements, while abundant, are known to limit the depth of analysis in blood proteomics and are considered detrimental to proteomic assays²³. However, their consistent reproducibility, after being confirmed from thousands of samples, provides an opportunity to enhance data analysis automation. This consistency enables more precise peak-picking for peptide and molecule targets without the need for spiking in exogenous standards.

### Evaluation of the Analytical Performances of the Protein Targeted Assays of the Complete360 Platform

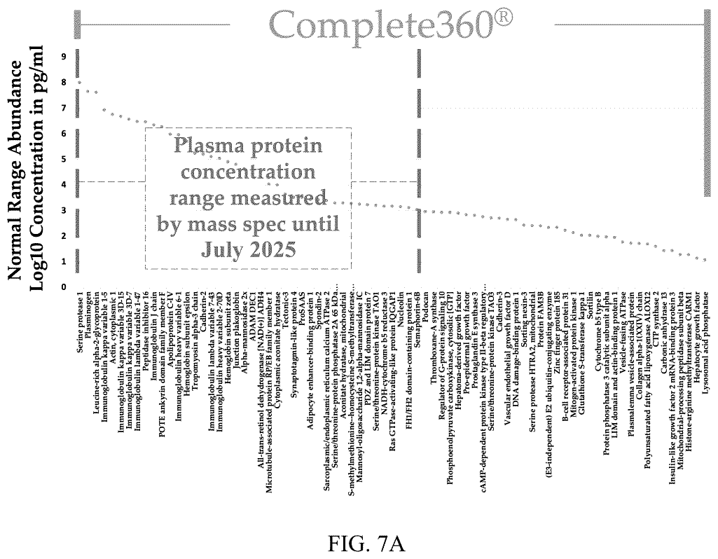

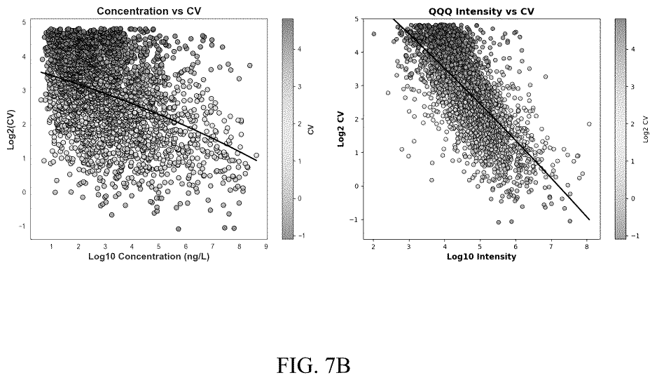

Proteins in plasma exhibit a vast dynamic range of concentrations, which presents a significant challenge in achieving the desired analytical depth for clinical proteomics assays. To evaluate the dynamic range of detectability provided by the Complete360 targeted assays, Applicants selected a small panel of well-documented plasma proteins with known concentrations. The findings reveal that the Complete360 assays enables the detection of plasma proteins within a remarkable concentration range—from ˜10 pg/mL to ˜100 ug/mL. This dynamic range encompasses the physiological plasma concentrations of most known plasma proteins (FIG. 7A).

One of the most challenging aspects of mass spectrometry-based detection of plasma proteins is sensitivity, which depends on factors such as co-elution dynamics, contamination, and ion suppression. Detecting low-abundance protein targets has been a critical area of interest for plasma proteomics. Through repeated optimizations, Applicants have demonstrated the ability to push the detection limits of a standard QqQ instrument to unprecedented levels at pg/mL (FIG. 7A), enabling the detection of extremely low-abundance targets^{6, 10-13, 24}. To further evaluate the detectability of Complete360 for extremely low-abundance plasma proteins, Applicants conducted assays using pooled plasma samples from healthy individuals described in previous studies⁶. The proteins detected were annotated with plasma concentration data from the Human Proteome Project (HPP) database and commercial assay providers. Notably, the lowest annotated protein detected by Complete360 was identified at a concentration of 3.5 pg/mL. Furthermore, numerous proteins in the sub-10 pg/mL range exhibited robust peak profiles on the Complete360 pipeline, underscoring its capability to detect even lower-abundance targets. For example, the protein Isocitrate dehydrogenase [NAD] subunit beta, mitochondrial (Uniprot ID: O43837) with a reported plasma concentration as 8.3 pg/mL was detected in different plasma samples at high abundance, covering various disease conditions. Despite its reported pg/mL level concentration, the signal intensity for this protein observed from Complete360 was strong that it remained detectable after a 1:1000 dilution (data not shown). These findings also indicate that Complete360 may achieve excellent detection performance even with further reduced sample input volumes. This feature has the potential to enable novel applications, such as in-home sample collection. Beyond Isocitrate dehydrogenase, several other low-abundance proteins demonstrated similarly strong intensity, including Leukocyte cell-derived chemotaxin 1 (UniprotID: O75829), NADH dehydrogenase [ubiquinone] flavoprotein 2, mitochondrial (UniprotID: P19404), Calretinin (UniprotID: P22676), and Methionine aminopeptidase 1 (UniprotID: P53582), etc.

Reproducibility is a critical requirement for clinical diagnostics, as it ensures precision and reliability in assay results. With clinical applications as the focus, Applicants systematically evaluated the reproducibility of the Complete360 assay using two complementary approaches:

Applicants evaluated the detection and quantification of 36 proteins across 12 replicates. These proteins span a wide dynamic range of documented plasma concentrations, from 19 pg/mL to 25 ug/mL, covering over 10⁶ orders of magnitude. By including proteins at varying concentrations in plasma, Applicants carefully assessed reproducibility across a wide dynamic range, highlighting the robustness and consistency of the Complete360 platform. Remarkably, the results demonstrated high reproducibility, with an average coefficient of variation (CV) in quantification of only 3.92% ranging from 1.4% to 7.0% across all proteins on the panel. Representative raw spectra of key proteins illustrate this exceptional reproducibility. These findings demonstrate that the Complete360 assay is highly reproducible, meeting the stringent requirements necessary for clinical applications.

To assess a broader range of proteins, Applicants conducted Complete360 assays targeting 9,977 proteins across five replicates. The median CV for the entire panel was 11.97%. When focusing on proteins with CVs below 25%, 7,833 proteins were consistently detected across all replicates, with a median CV of 8.73%. Notably, 4,361 proteins exhibited CVs below 10%, with a median CV of 4.77%. This subset of highly reproducible proteins demonstrates significant potential for direct translation into clinical applications once a clinical relevance to a disease is validated.

It is noteworthy that the reproducibility of protein abundance measurements and signal intensity detected by the Complete360 platform demonstrates a strong correlation (FIG. 7B). As the biological concentration of proteins in plasma increases, the reproducibility of detection improves accordingly. Moreover, the Complete360 platform reliably quantifies protein targets across a dynamic range exceeding eight orders of magnitude. The correlation between QqQ intensity and reproducibility is remarkably high, with an R² value of 0.51. It is also important to highlight that only about 4,500 proteins have documented blood concentration data to date²⁵. Interestingly, most of the proteins included in the Complete360 full panel are not yet documented for their blood concentration levels. Future efforts will focus on systematically establishing these concentration profiles to further enhance the clinical and diagnostic utility of the platform, including achieving reliable absolute quantifications.

Next, Applicants compared the reproducibility and detectability of Complete360 to conventional DIA-based proteomic profiling. Using timsTOF HT to analyze the same sample, Applicants conducted three technical replicates, resulting in the identification of 7,697 proteins using DIA-MS. Among these, 3,944 proteins were consistently detected across all three replicates, 2,737 proteins were observed in two out of three replicates, and 1,016 proteins were identified in only one replicate. For proteins detected in two or more replicates, the median CV for quantification was calculated to be 15.23%, indicating a high-quality profiling assay. These results demonstrate that Complete360, with its optimized parameters, provides substantially enhanced reproducibility and quantification consistency compared to conventional DIA-based approaches.

The findings demonstrated the reproducibility of the Complete360 platform across both small- and large-scale protein panels, covering a wide dynamic range of protein concentrations. The ability to consistently achieve results with CVs well within acceptable thresholds highlights Complete360 as a reliable and practical solution for clinical applications. Its enhanced reproducibility and sensitivity, offering improved reliability and confidence in detection and quantification plasma proteins, making Complete360 a valuable tool for mass spectrometry-based proteomics, particularly in clinical diagnostics where precision and consistency are essential.

### Disease-Associated Molecular Changes Revealed by Complete360

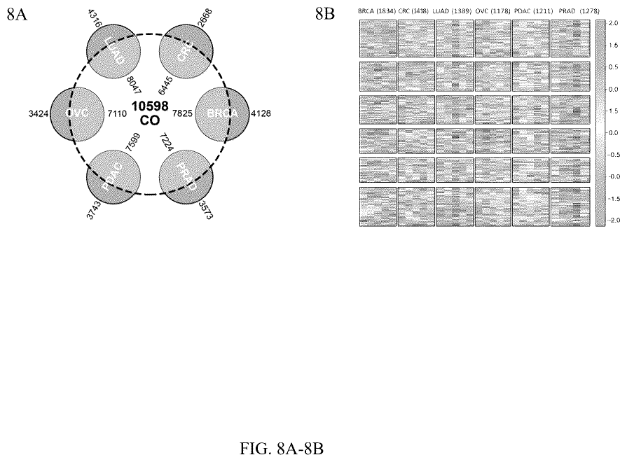

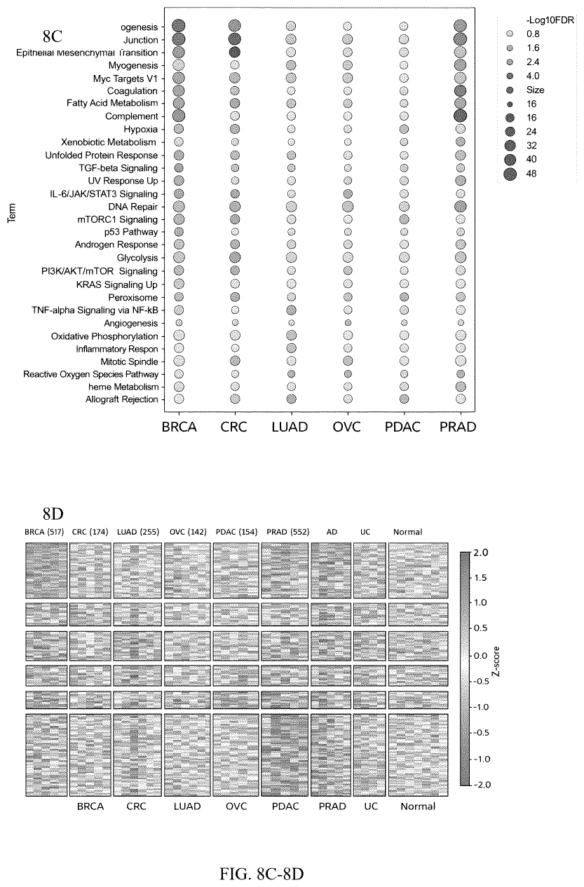

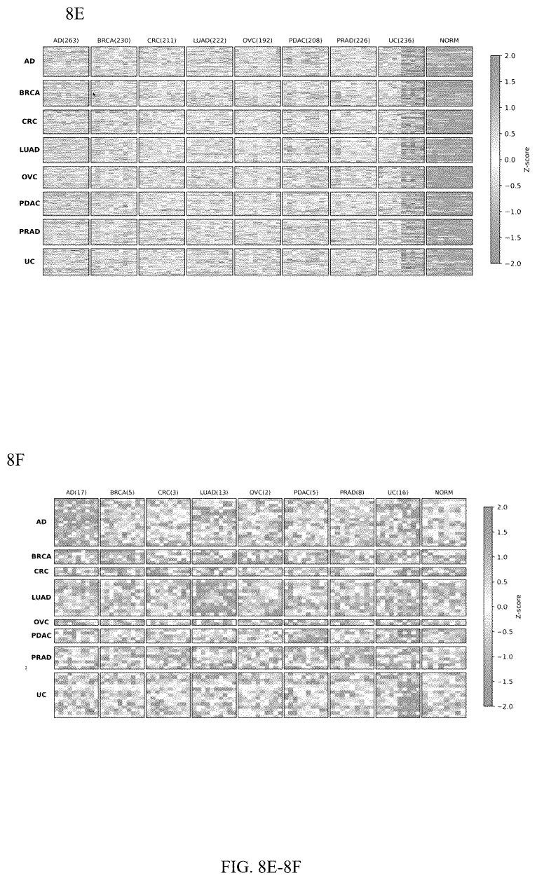

To investigate the clinical utilities of the Complete360 platform, Applicants applied the Complete360 assays to the analyses of plasma samples from individuals with different diseases, representing patients diagnosed with breast cancer (BRCA), colon cancer (CRC), lung adenocarcinoma (LUAD) , ovarian cancer (OVC), pancreatic adenocarcinoma (PDAC), prostate adenocarcinoma (PRAD), Alzheimer's disease (AZ), Ulcerative Colitis (UC), and non-disease control (Normal). In total, 10,598 proteins were quantified from the normal and disease samples (FIG. 8A). Comparing these proteins with the proteins identified from cancer tissues from the Clinical Proteomic Tumor Analysis Consortium (CPTAC) and previous reports for BRCA, CRC, LUAD, OVC, PDAC, and PRAD, majority of proteins identified from these tumor tissues could be quantitative analyzed in plasma using Complete360 assays, including 7,825 BRCA tumor proteins in plasma, 7,224 tumor tissue proteins from PRAD in plasma, 7,599 tumor tissue proteins from PDAC in plasma, 7,110 tumor tissue proteins from OVC in plasma, 8,047 tumor tissue proteins from LUAD in plasma, and 6,445 tumor tissue proteins from CRC in plasma (FIG. 8A)²⁶⁻³⁶. The results indicated that these disease-associated proteins, once identified from disease tissues from the discovery studies, could be readily quantified in patient plasma for disease detection or treatment monitoring using blood tests.

Among the plasma detectable tumor proteins (FIG. 8A), significant portions of proteins are over-expressed (FC at least 2-fold in plasma from different cancer types comparing to normal individuals, leading to the identification of 1,834 BRCA, 1,418 CRC, 1,389 LUAD, 1,178 OVC, 1,211 PDAC, and 1,872 PRAD tumor proteins that were overexpressed in plasma (FIG. 8B). This data showed that a significant disease-associated tissue proteins were differentially expressed in plasm samples from disease plasma comparing to plasma proteins from normal group due to the sensitive and quantitative nature of the Complete360 assays.

To uncover the molecular programs related to the different cancer-associated plasma proteins, Applicants investigated the identified proteins using over-representation analysis (ORA) via GSEApy referencing Hallmark gene sets. While the analysis revealed several commonly known cancer Hallmarks gene sets enriched in the elevated plasma proteins for different cancer types, including apical junction, epithelial mesenchymal transition, myc targets V1, fatty acid metabolism, hypoxia, mTORC1 signaling, p53 pathway, and glycolysis, cancer-specific activations of distinct biological programs for elevated cancer proteins were also observed (FIG. 8C). BRCA was dominated by adipogenesis, myogenesis, complement, xenobiotic metabolism, and KRAS signaling up. CRC showed strong enrichment for xenobiotic metabolism, and DNA repair. LUAD showed major enrichment at TNF-alpha signaling and oxidative phosphorylation. OVC showed moderate enrichment in myogenesis and mitotic spindle. PDAC was associated with DNA repair and allograft rejection. PRAD was dominated by adipogenesis, myogenesis, coagulation, complement, xenobiotic metabolism, DNA repair, and heme metabolism. The results showed that both common and disease-specific molecular pathways were detected in plasma using the Complete360 assays.

The cancer-type specific proteins were further investigated on plasma samples from individuals with different diseases. These resulted in the identifications of 517, 174, 255, 142, 154, 552 tissue proteins with elevation as cancer type-specific proteins (FIG. 8D). This data showed that the targeted plasma proteomic assays contained disease-associated molecular changes. Both pan-cancer associated molecular and pathway changes and cancer-specific protein changes were presented in patient plasma samples, which were detectable using the Complete360 assays.

The analysis of normal and disease plasma using Complete360 multi-omic assays also identified and quantified 762 metabolites and 1,395 lipids through its MyMeta pipeline. Elevated metabolites for each disease type were identified by comparing the data from different disease groups to the normal control group for metabolites (FIG. 8E) and lipids. The disease-type-specific molecular changes were revealed for metabolites (FIG. 8F) and lipids. Multi-omic targeted analysis using Complete360 indicated that the targeted plasma assays for metabolites and lipids contain disease-specific and disease shared small molecule changes.

### Simultaneous Quantification of Plasma Proteins, Metabolites, and Lipids for Enhanced Diagnostic Precision

To achieve comprehensive diagnostics, Applicants have integrated proteomics, metabolomics and lipidomics analysis within the Complete360 platform, enabling the simultaneous detection and quantification of proteins, metabolites, and lipids from the same biological sample through the same platform. The current Complete360-MyProt assay utilizes a targeted approach to quantify 10,598 plasma proteins, representing the most comprehensive coverage of human plasma proteins reported to date. In parallel, the Complete360-MyMeta assay targets 762 polar metabolites and 1,395 lipids, offering broad and integrated coverage of metabolic and lipidomic pathways. Each assay has undergone a systematic multi-parameter and multi-cycle optimization to enhance sensitivity, reproducibility, and quantitation accuracy. This streamlined multi-omics approach maximizes sample utilization, enhances diagnostic accuracy while maintaining cost efficiency. By consolidating all assays onto a unified platform, the method facilitates seamless clinical implementation, supporting broader adoption in clinical and translational research.

For each disease, the integrated approach has yielded highly informative results, revealing matched metabolic and proteomic signatures. Notably, pathway analysis shows strong concordance between proteomic and metabolomic data, with an average of 69% of the top 10 pathways concerning metabolomics identified from each omics perspective overlapping. This alignment underscores the robustness and biological relevance of the integrated Complete360 platform in disease characterization.

To fully leverage the capabilities of the multi-omics Complete360 platform, Applicants integrated proteins, metabolites, and lipids as key features and performed a t-test to compare each disease against all other conditions. Molecules were ranked in ascending order based on p-values, and the top 1,000 features were consistently selected for model construction. Applicants then generated ROC curves using only the top 1,000 proteins for each disease and compared them to ROC curves generated when the top 1,000 features-comprising proteins, polar metabolites, and lipids-were collectively incorporated. To ensure consistency, the total feature count was always maintained at 1,000, allowing the model to determine the optimal composition of proteins, metabolites, and lipids for each disease-specific diagnostic panel. On average, 794 proteins were selected, while the mean feature count for metabolites and lipids was 90 and 117, respectively. Notably, Applicants observed an increase in AUC values for most diseases when multi-omics features were incorporated into the diagnostic model, underscoring the advantage of integrating multiple molecular layers.

This enhancement highlights the unique value of the Complete360 platform, which enables the simultaneous and cost-effective analysis of multi-omics analytes on a single instrument, improving both diagnostic accuracy and efficiency. Applicants acknowledge that the current study is based on a limited sample size, and while the observed ROC curves provide valuable insights, they may not fully capture the diagnostic potential of the approach. Future studies with larger sample cohorts and deeper data analysis approaches will be conducted to further validate these findings, as this work primarily serves as a proof-of-concept demonstration of the Complete360 platform.

### Disease-Specific Proteins by Complete360 Assays Used to Detect Disease in Plasma

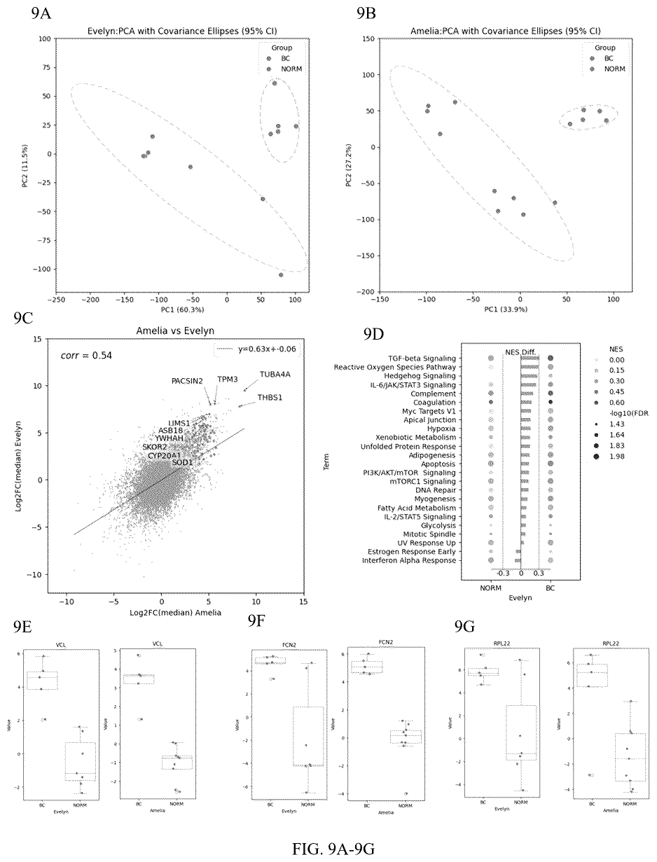

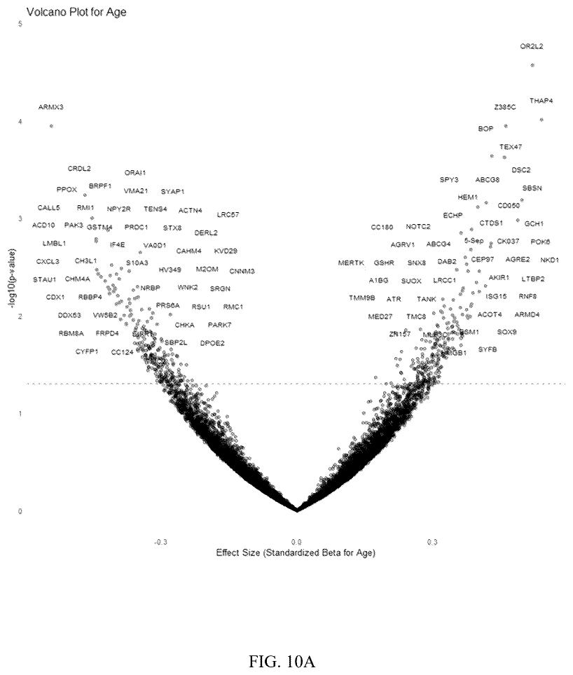

The disease-specific proteins detected in plasma by Complete360 could be used to detection disease in plasma. The comparison between the plasma proteins from breast cancer and non-tumor samples using the Complete360 assays could lead the discovery of specific plasma protein changes for breast cancer. The significantly altered plasma proteins between tumor and non-tumor samples could be potentially useful for the diagnosis of breast cancer. In the discovery plasma proteomic data set using Evelyn MS instrument, the principal component analysis (PCA) of breast cancer (n=5) and non-cancerous samples (n=7) illustrated a formation of distinct clusters of the cancer and non-cancerous samples (FIG. 9A). All cancer samples were differentiated from the non-cancerous samples. From the differential analyses of breast cancer and non-cancerous control plasma samples, Applicants identified 331 up-regulated and 284 down-regulated plasma proteins. To verify the identified plasma protein changes, Applicants analyzed additional plasma samples from 5 independent breast cancer patients and 9 non-cancerous plasma samples by Complete360 assays with an orthogonal MS instrument, Amelia. The PCA analysis showed the formation of distinct clusters of the cancer and non-cancerous samples (FIG. 9B). Applicants found that the quantitative data from the independent patient plasma with orthogonal MS instrument is consistent with the discovery analysis with correlation of 0.54 (FIG. 9C). Using the verification breast cancer data, 105 of the 331 elevated plasma proteins identified from discovery data were verified by the independent analyses of the additional samples using Complete360 assays with a different MS instrument.

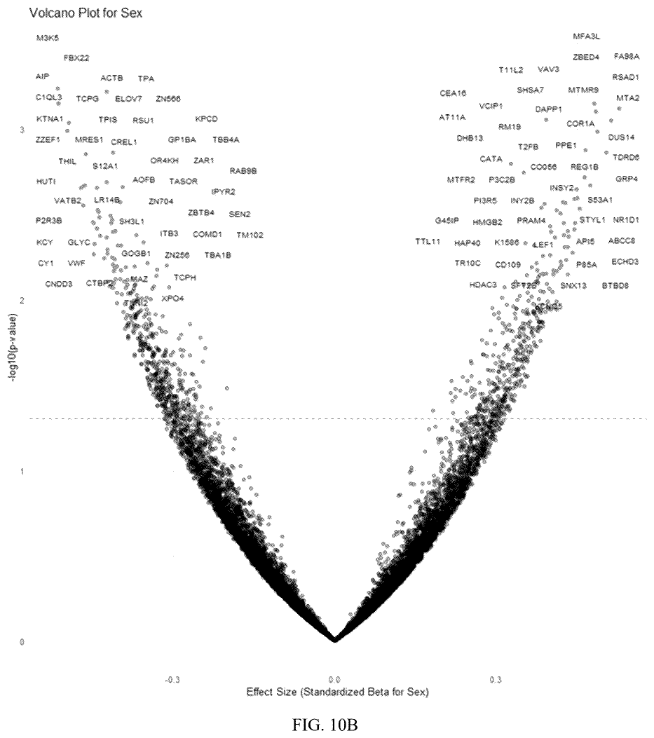

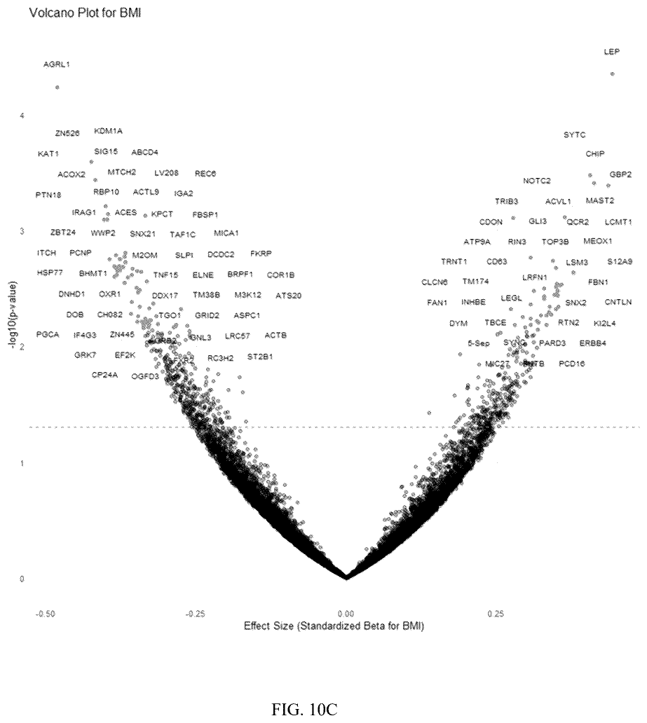

To investigate the classes of plasma proteins that were specifically regulated in breast cancer, the Hallmark pathways were performed on the significantly positive regulated plasma proteins (FIG. 10D) and revealed that TGF-beta signaling, reactive oxegen species pathway, IL-6/JAK/STAT3 signaling, complement, Myc targets are top overrepresented pathways for plasma proteins elevatged in breast cancer, while estrogen responses early, interferon alpha response were overrepresented in significantly down-regulated proteins for breast cancer.

The comparison between the plasma proteins from breast cancer and non-cancerous samples could lead the discovery of specific protein changes for breast cancer and serve as protein markers for breast cancer detection using blood samples. Among the upregulated plasma proteins in breast cancer plasma, several key proteins linked to tumor progression and metastasis were identified. Notable proteins include vinculin (VCL, FIG. 9E), involved in cell migration and metastasis^{37, 38}. Ficolin-2 (FCN2, FIG. 9F), an immune system protein, plays a role in immune surveillance, and Large ribosomal subunit protein eL22 (RPL22, FIG. 9G), frequently deregulated in cancers, suggests its role in tumor biology^{39, 40}.

### Plasma Proteome Variation and Its Genetic Determinants Revealed by Complete360

Using Complete360 methods, Applicants have conducted an ultra-deep plasma proteomics analysis to investigate the correlation between plasma protein levels and human age, gender, and BMI. The findings align closely with those reported by Mann et al., demonstrating that a significant proportion of the plasma proteome varies systematically with these demographic and physiological factors⁴¹. Notably, Applicants identified age-, gender- or BMI-associated shifts in proteins involved in inflammation, extracellular matrix remodeling, lipid metabolism, and coagulation cascades, reflecting the dynamic changes in systemic physiology (FIG. 10).

Complete360's high-sensitivity protein detection enabled the identification of BMI-associated proteomic signatures, uncovering key proteins involved in metabolic regulation, inflammatory response, and lipid transport (FIG. 10). Notably, proteins such as TRIB3 (a regulator of obesity and insulin resistance), INHBE (a determinant of fat distribution), and ERBB4 (which modulates brain-regulated energy expenditure) exhibited distinct expression profiles across BMI categories. Among these, LEP (leptin) emerges as a particularly significant contributor to BMI, reinforcing its well-documented role in weight regulation42. These findings reveal a robust molecular signature of metabolic health, providing a valuable framework for biomarker discovery and disease risk stratification. The strong correlation between these plasma proteins and BMI underscores the predictive power of Complete360 in distinguishing metabolic states. This highlights the potential of plasma proteomics not only as a biological clock for metabolic health but also as an innovative tool for early disease detection and personalized health monitoring.

Furthermore, Complete360 facilitates genetic-proteomic association studies (pQTL analysis) to determine the genetic influences on plasma protein levels. The initial findings suggest that genetic variants contribute significantly to the observed protein-level variance, with some proteins showing strong cis- and trans-regulatory effects. The integration of Complete360 with genome-wide association studies (GWAS) is expected to further uncover causal relationships between genetic factors, proteomic alterations, and disease predisposition.

With its ability to quantify thousands of plasma proteins at high specificity, capture proteomic variability with minimal technical noise, and support predictive modeling of age and BMI, Complete360 is at the forefront of precision medicine and multi-omics biomarker research. These insights will be instrumental in improving disease risk assessment, enhancing therapeutic targeting, and advancing the understanding of human health at the molecular level.

### Discussion

Complete360 is a highly targeted and comprehensive detection platform, capable of quantifying close to 13,000 molecules from blood samples, delivers unmatched sensitivity and reproducibility, exceeding the capabilities of traditional profiling methods commonly used in academic and clinical settings. Underpinned by the comprehensive CompleteBank databases and CompletePeaking algorithms, Complete360 establishes itself as a potential transformative tool for basic research and clinical diagnostics, offering advancements in biomarker discovery, disease pathway analysis, and personalized medicine. It is designed to bridge the gap between multi-omics research and real-world clinical applications, enabling newly identified molecular changes to be seamlessly translated into clinical use on the same platform.

Proteomics assays generally follow two main approaches: mass spectrometry-based methods and affinity-based detection techniques. Mass spectrometry relies on advancements in instrumentation, sample preparation, and data analysis algorithms, while affinity-based methods employ antibodies or aptamers to facilitate assays such as ELISA or its variations, like proximity extension assays (PEA). Although affinity-based methods have been widely applied to clinical specimens, their limitations are apparent. They rely heavily on the quality of the binding reagents, which can lead to inconsistencies due to variations in the manufacturing of antibodies or aptamers⁴³⁻⁴⁵. Even with high-quality binding reagents, the detection may be hindered by the limited accessibility of epitopes; many proteins in blood are modified by different protein modifications or form complexes by binding to other molecules, obscuring their binding sites⁴⁶. Furthermore, many proteins that serve as valuable biomarkers and are involved in rapid physiological responses have relatively short half-lives therefore hindering their detection by affinity-based methods^{47, 48}. For example, insulin and glucagon, both critical for glucose regulation, have half-lives of about 5 to 10 minutes, while cytokines like interleukin-6 can range from minutes to a few hours. Although these short-lived proteins are essential disease biomarkers, accurately detecting them using affinity-based methods is challenging. This is due to epitope masking through binding to other proteins, or rapid epitope damage and degradation caused by protease digestion. These factors often result in compressed fold-change data in affinity-based methods, making it difficult to differentiate between disease and healthy individuals. As a result, the sensitivity and specificity required for effective diagnostics are significantly compromised.

Mass spectrometry-based proteomics offers superior specificity and resolution, making it well-suited for distinguishing disease from control samples. However, these methods also face challenges related to sensitivity and reproducibility. Most proteomics assays employ profiling techniques using orbitrap or time-of-flight mass analyzers, and these platforms often fall short of the reproducibility standards required for clinical applications, where a coefficient of variation (CV) below 10% is essential. While triple quadrupole mass spectrometers are widely used in clinical laboratories to detect disease-associated small molecules, they require extensive optimization of detection parameters, including sample preparation strategies. Despite efforts to establish standardized detection protocols using synthetic peptides, these parameters often remain theoretical and may not fully account for the variability of real-world clinical samples.

Given these challenges, there is an urgent need for a robust and reproducible proteomics platform capable of detecting a broad spectrum of clinically relevant proteins and metabolites from blood samples with high accuracy and reliability. This is the driving force behind the development of Complete360. The platform is designed to provide a finely tuned, clinically viable system for comprehensive proteomic and metabolic analysis in blood, ensuring the reproducibility and sensitivity required for clinical applications. Through years of refinement, Applicants have optimized sample preparation workflows, established precise detection parameters, and developed a sophisticated data analysis pipeline. Validated through the analysis of a good amount of body fluid samples, Complete360 represents a major advancement in proteomics research and its translation into clinical practice. Looking ahead, Applicants aim to extend the application of Complete360 beyond basic research to direct clinical diagnostics. The goal is to implement this platform across multiple countries, facilitating improved disease detection and better patient outcomes. By bridging the gap between proteomics research and clinical application, Complete360 has the potential to redefine the future of precision medicine.

### Materials and Methods

### Chemicals and Reagents

For blood proteomics: Water Optima LC/MS grade; Acetonitrile Optima LC/MS grade; Methanol LC/MS grade; Ammonium Bicarbonate (ABC) 1M; Tris buffered saline (Sigma), Formic Acid 98%-100%; Sodium dodecyl sulfate (SDS); Tris-(2-Carboxyethyl)phosphine-HCl (TCEP); 2-Chloroacetamide (CAA) ≥98%; Triethylammonium bicarbonate (TEAB) 1.0 M; Triethylamine; Phosphoric Acid; Promega sequencing grade Trypsin; Whatman 903™ blood collection kit, Minute™ albumin depletion kit.

For small molecules: Ammonium formate, Ammonium acetate, Ammonium hydroxide solution: Sigma-Aldrich; Methanol (LC), water (LC/MS Grade), acetonitrile, and 2-propanol (LC/MS grade, LiChrosolv): Fisher Sci.

### Patients and Samples

Plasma samples used in this study were obtained from BioIVT (Westbury, NY, USA), along with comprehensive clinical information. All samples were collected in accordance with institutional ethical guidelines and were de-identified to ensure patient confidentiality. Plasma was collected using purple-top tubes containing EDTA as an anticoagulant. Upon collection, samples were processed promptly by centrifugation to separate plasma, aliquoted, and stored at −80° C. until further use to minimize freeze-thaw cycles and maintain proteomic and metabolic stability.

### Plasma Sample Preparation and Analysis Methods

Plasma samples were processed using the Complete360-MyProt pipeline, incorporating the proprietary Chemical-Biological Plasma Protein Preparation procedure. This workflow starts from two key steps to remove high abundance proteins and collect clinically and biologically more meaningful low-abundance proteins:

Chemical Procedure: Major plasma proteins were precipitated using a set of in-house-prepared protein removal reagents.

Biological Procedure: The remaining high-abundance and median-abundance plasma proteins were depleted using a proprietary antibody-conjugated resin, targeting a combination of proteins that are most frequently detected by mass spectrometry and reported in the database of peptide atlas. Such depletion procedure has been observed to be more reproducible for protein quantifications compared to that of nanoparticle-based plasma low-abundance protein enrichment methods (data not shown). Protein depletion methods for other body fluid can be established the same way. The depletion resin is tested for durability, demonstrating consistent performance for over 200 uses with optimized buffers and procedures (data not shown) to ensure an ultra-low cost for plasma protein extraction.

After the removal of high- and median-abundance proteins through this chemical-biological procedure, the plasma proteins remaining in the supernatant were processed into peptides using the Complete360 sample digestion kits and reagents. Briefly, plasma proteins were denatured using SDS and digested with an optimized trypsin digestion protocol. Following digestion, the resulting peptide samples were fractionated using an offline HPLC system operating in both low-pH and high-pH modes. This dual-mode approach ensures highly reproducible chromatographic profiles. The procedures were extensively optimized for human plasma samples, with key metrics such as protein identification, detected abundance, and mis-cleavage rates carefully monitored to ensure reproducibility and sensitivity of mass spectrometry analysis.

For proteomics analysis using dried blood spot (DBS) samples, three 12 mm disks were pooled and incubated in Tris-buffered saline (TBS) containing 0.05% NP-40 at 37° C. for 30 minutes with agitation. The supernatant was then combined with an equal volume of the Minute™ Albumin Depletion Kit reagent to remove albumin and hemoglobin; this depletion step was repeated twice. The resulting precipitate was solubilized in TBS and subjected to digestion using Complete360 sample digestion kits and reagents as described above.

Peptide digests were then subjected to basic reversed-phase chromatography using an Agilent 1260 liquid chromatography system, following the methodology outlined reported previously⁴⁹. Separation was performed on an in-house packed C18 column employing a gradient of acetonitrile in 10 mM triethylammonium bicarbonate (TEAB). The gradient conditions were as follows: 5% to 28% solvent B over 75 minutes, increased to 42% over the next 8 minutes, and then to 98% over the subsequent 3 minutes, at a flow rate of 1 mL/min. Fractions were collected every minute and were then concentrated to dryness using a Speed Vac equipped with a chilled vacuum trap. The dried peptides were stored at −80° C. until further analysis.

DIA-MS Analysis: Mass spectrometric discovery analyses were conducted using a timsTOF HT mass spectrometer coupled to a nanoElute 2 liquid chromatography system via a CaptiveSpray™ ion source, configured in a two-column setup comprising a 5 mm Thermo trap cartridge and a PepSep Max Ten series analytical column (10 cm×150 μm i.d., 1.5 μm particle size).

DDA-PASEF Analysis: To assess the quality of trypsin digestion, including the evaluation of missed cleavages and potential artifacts, data-dependent acquisition parallel accumulation-serial fragmentation (DDA-PASEF) analyses were performed. This approach facilitated the identification of peptides and proteins, ensuring the integrity of the digestion process.

DIA-PASEF Analysis: For comprehensive proteomic profiling, data-independent acquisition PASEF (DIA-PASEF) analyses were executed. The acquisition method was optimized to minimize missing data and to cover ion mobility ranges with high-density precursor sampling. The method consisted of eight cycles, each comprising 29 ion mobility (IM) windows. An initial MS1 scan was followed by eight DIA-PASEF cycles, covering an m/z range of 375-1100 and an inverse reduced mobility (1/K₀) range of 0.65-1.45 V·s/cm². The resulting DIA-MS data were processed using DIA-NN (version 1.8.2) employing a predicted human protein spectral library containing 20,480 entries. Both DDA- and DIA-PASEF datasets were analyzed using DIA-NN and FragPipe software to ensure comprehensive identification and quantification of peptides and proteins^{50, 51}.

Complete360-MyProt Analysis: Targeted detection and quantification for plasma proteins was performed on an Agilent 6495 QqQ Mass Spectrometer using dynamic Selected Reaction Monitoring (dSRM) with an Agilent Jet Stream ion source. Chromatographic separation was achieved using an in-house packed C18 column (1.7 μm, 2.1 mm×30 mm). The gradient of solvent B (acetonitrile with 0.1% formic acid) was programmed as follows: 12% to 42% over 5.4 minutes, followed by an increase to 98% in the next minute, at a flow rate of 150 μL/min. A set of targeted assays were created through CompleteBank-Discovered and CompleteBank-Validated process to therefore detect and quantify over 10,000 plasma proteins in the same assay.

### Sample Preparation and Analysis Methods for Small Molecules

Plasma samples were processed using the Complete360-MyMeta pipeline using a modified MTBE/MeOH/H2O extraction protocol to isolate lipids and polar metabolites from 40 ul plasma sample. First, 300 μL of cold methanol was added to the plasma aliquot and vortexed for 10 seconds. Following this, 1 mL of methyl tert-butyl ether (MTBE) was added, and the mixture was vortexed again for 10 seconds before being incubated on a shaker at room temperature for 60 minutes. After incubation, 250 μL of water was added to induce phase separation, followed by a 10-minute incubation at room temperature with occasional vortexing. The samples were then centrifuged at 15,000 g for 10 minutes to separate the phases. The upper organic lipid phase (approximately 900 μL) was collected into a clean 2 mL glass vial, while the lower aqueous metabolite phase (320-350 μL) was also collected. The organic phase was dried under nitrogen gas for 20-30 minutes and reconstituted in 300 μL of 1-butanol/methanol (1:1) containing 10 mM ammonium formate. The aqueous phase was dried under nitrogen and reconstituted in 150 μL of 50% acetonitrile (ACN).

Complete360-MyMeta Analysis: For MyMeta analysis, metabolites were separated on an Agilent 1290 LC system using two HILIC-LC methods. The first method, HILIC-01, employed an Acquity BEH-Amide column (1.7 μm, 2.1×150 mm) at a column temperature of 40° C. and an autosampler temperature of 8° C. The injection volume was 5 μL, with mobile phase A consisting of 95% water +20 mM ammonium acetate (pH 9.4), and mobile phase B being 98% acetonitrile. The flow rate was set at 0.15 mL/min with a gradient program running as follows: 0 minutes, 90% B; 2 minutes, 90% B; 3 minutes, 75% B; 7 minutes, 75% B; 8 minutes, 70% B; 9 minutes, 70% B; 10 minutes, 50% B; 12 minutes, 50% B; 13 minutes, 25% B; 14 minutes, 25% B; 16 minutes, 0% B; 20 minutes, 0% B; 21 minutes, 90% B; and 25 minutes, 90% B, with a 2-minute post-run period. The second method, HILIC-02, used an Atlantis Premier BEH Z-HILIC column (1.7 μm, 2.1×100 mm) at a column temperature of 30° C. and an autosampler temperature of 8° C. The injection volume was again 5 μL, with mobile phase A consisting of 70% water +5 mM ammonium formate (pH 4.0) and mobile phase B composed of 95% acetonitrile +5 mM ammonium formate (pH 4.0). The flow rate was set to 0.25-0.4 mL/min, with the gradient program as follows: 0 minutes, 100% B (flow rate 0.25 mL/min); 1 minute, 100% B (flow rate 0.25 mL/min); 10.5 minutes, 60% B (flow rate 0.25 mL/min); 13 minutes, 15% B (flow rate 0.25 mL/min); 13.5 minutes, 15% B (flow rate 0.25 mL/min); 14 minutes, 100% B (flow rate 0.4 mL/min); 18.5 minutes, 100% B (flow rate 0.4 mL/min); 19 minutes, 100% B (flow rate 0.25 mL/min); 20 minutes, 100% B (flow rate 0.25 mL/min).

Targeted detection and quantification for plasma metabolites and lipids analysis was performed on an Agilent 6495 QqQ Mass Spectrometer using dynamic multiple reaction monitoring (dMRM) with an Agilent Jet Stream ion source. The polarity was switched between positive and negative modes. The gas temperature was set to 200° C., with a drying gas flow rate of 14 L/min, nebulizer gas at 50 psi, and sheath gas at 375° C. and 12 L/min. The capillary voltage was set to 3,000 V for positive and −2,500 V for negative polarity, with a nozzle voltage of 0 V for both polarities. The iFunnel high/low pressure RF was set to 150/60 V for positive and 90/60 V for negative polarity. The scan type was set to dMRM with unit resolution for both Q1 and Q2, a delta EMV of 0 V (positive) and 200 V (negative), and a cell acceleration voltage of 5 V. The dMRM method was generated using Agilent MassHunter Acquisition software.

### Development of Plasma Proteomics Fingerprint Database: CompleteBank-Discovered

For Plasma Proteins: Applicants analyzed over 9,000 plasma proteomic runs using the timsTOF HT mass spectrometer (Billerica, Massachusetts, USA), systematically documenting the performance of each detected plasma protein. A stringent false discovery rate (FDR) threshold of 1% was applied in discovery-mode analysis to ensure high confidence in protein identification. By integrating data from these runs, Applicants identified 17,328 unique plasma proteins. To facilitate validation on triple quadrupole (QqQ) mass spectrometers, Applicants categorized these proteins into distinct classes based on their physicochemical properties and detection characteristics. A robust algorithm was developed to effectively translate protein fingerprints identified on the time-of-flight (TOF) platform to QqQ mass spectrometers. Validation assays were subsequently conducted using QqQ platforms from multiple vendors to confirm the reproducibility and reliability of these identified proteins.

Plasma Metabolites and Lipids: To establish a comprehensive plasma metabolite and lipid profile, Applicants compiled a curated list of nearly 3,000 small molecules, including metabolites and lipids, based on an extensive literature review. Each molecule was subjected to at least six different analytical approaches to determine the optimal detection conditions in human plasma samples. To maximize the number of detectable small molecules while minimizing assay run time and improving throughput, Applicants manually curated and optimized the resulting data. This refinement process led to the development of a high-efficiency detection protocol, ultimately documenting 2,927 molecules in the CompleteBank-Discovery database.

### Establishment of Validated and Optimized Detection Assay Clusters: CompleteBank-Validated

To establish the targeted detection assay clusters, there are several key considerations:

Separation of high- and low-abundance molecules: High- and low-abundance molecules should be detected separately to minimize ion suppression effects on low-abundance targets. This ensures that low-abundance molecules are detected with enhanced sensitivity and reproducibility, avoiding interference from high-abundance molecules, which typically overshadow other target analytes²³.

Resolution of co-eluted targets: Co-eluted targets should be resolved at higher resolution when their abundance permits. While higher resolution may reduce the number of ions entering the mass analyzer of a mass spectrometer, potentially affecting sensitivity, careful manual tuning and optimization for each protein can address this.

Retention time reproducibility: Retention times for various analytes must be consistently reproducible across different assay environments. For instance, an analyte detected in a low pH fraction may exhibit a significantly different retention time compared to its detection in a high pH fraction. This variation underscores the importance of considering the surrounding microenvironment to ensure reproducible detection schedule.

Management of co-eluting noise analytes: High-abundance noise analytes must be carefully controlled to avoid co-elution with low-abundance target analytes. In the assay, some high-abundance analytes have been utilized to serve as highly reproducible landmarks, which can enhance data annotation accuracy and improve overall analytical precision which is further illustrated later.

Minimizing Run Time: To integrate all target analyses into a single assay with minimal runtime, an AI-driven pattern-clustering approach was employed. This method reduced overall run time while optimizing the sensitivity and reproducibility of each analyte when consolidated into the same assay. This AI-driven algorithm is an essential component of the in-house developed software package, which can also be used to establish customized diagnostic methods, such as those focused on specific diseases.

Therefore, Applicants established the following two sets of methods, one set is targeting a list of 10,598 proteins and the other one is targeting a list of 2,157 polar metabolites and lipids.

For Plasma Proteins: To transition from discovery to validated clinical applications, Applicants evaluated the clinical relevance of each identified plasma protein and selected an initial panel of 12,892 proteins from the discovery cohort. Extensive QqQ-based method optimization was performed for these proteins, involving iterative assays to fine-tune detection conditions, enhance sensitivity, and ensure reproducibility. Through this rigorous process, Applicants refined the panel to a final set of 10,598 validated proteins, optimized for reliable quantification using QqQ mass spectrometry.

Plasma Metabolites and Lipids: For targeted metabolomics and lipidomics, Applicants established validated detection parameters for 762 metabolites and 1,395 lipids. To achieve optimal characterization, Applicants employed three different chromatographic columns and implemented six distinct analytical methods, ensuring comprehensive coverage and precise quantification of these molecules.

### CompletePeaking: An Automatic Bioinformatic Pipeline for Clinical Proteomics and Metabolomics Data Processing

For Plasma Proteins: One of the essential parts of the Complete360 pipeline is its methodology for peak picking and data analysis pipeline, and Applicants term this entire bioinformatic pipeline as CompletePeaking. The CompletePeaking process combines manual curation with automated machine learning approaches to ensure the accurate identification and quantification of peptides in complex proteomic datasets. Initially, Applicants employed the Complete360 methods for manual validation of proteomics data, where human evaluators examined chromatograms and spectra for peptide transitions. The goal was to ensure that the peaks of interest coeluted with reference transitions and exhibited minimal background noise. Manual validation focused on assessing coelution of peaks and verifying that they exhibited high library dot product scores, which quantify the similarity between observed and reference intensity profiles.

Each peptide transition was scrutinized for quality indicators, including signal-to-noise ratio (SNR), intensity, and the presence of coelution, with a tolerance threshold of 0.3 minutes for retention time, as well as surrounding noise signals for each specific transitions. The manual curation process, although labor-intensive, was crucial in establishing the initial training dataset across diverse clinical samples, including several advanced stage cancer plasma samples, cardiovascular disease samples, neurodegenerative disease plasma samples and also inflammation and auto-immune disease plasma samples. These curated datasets formed the foundation for subsequent CompletePeaking model training.

Validation Process: During the manual validation phase, evaluators closely monitored the retention times of peptide transitions to ensure they coeluted. The measured retention times were then compared to predicted peak retention times, ensuring they fell within a defined tolerance. Dot product calculations were performed to assess the similarity between the observed and reference spectra, with high dot product scores indicating a strong match to the expected peptide identity. Peaks exhibiting both strong coelution and high dot product scores were labeled as “good” peaks, which were subsequently used as input for model training.

Despite the advantages of manual validation, it introduces certain biases. Evaluators often tend to prioritize peaks with higher intensity, which, while visually striking, may not always correspond to biologically relevant transitions. To mitigate these biases, Applicants incorporated several strategies to improve the reliability of the training data. These included matching retention times across multiple samples, identifying reproducible noise landscapes around target peaks, and closely examining the patterns of transition similarity to the reference library. This comprehensive approach helped to ensure more accurate and consistent peak selection, enhancing the reliability of the training data.

A critical aspect of this study is the long-term accumulation of data, with years of manual peak validation forming the core of the training dataset. By manually curating and validating a large number of peaks across diverse clinical samples, Applicants developed a robust dataset that captured the nuances of peak coelution, retention times, and library dot product scores. This curated dataset served as the backbone for training machine learning models, enabling the automation of peak selection while preserving the high accuracy and reliability achieved through manual methods.

Automated Peak Picking Using Machine Learning: To address the limitations of manual peak selection and improve scalability, Applicants integrated an artificial intelligence-based machine learning model to automate the peak picking process. The model was trained using the XGBoost algorithm with a dataset that had been manually validated, incorporating various features derived from chromatogram analysis, such as coelution count, signal-to-noise ratio (SNR), shape correlation, and intensity. These features were essential for distinguishing high-quality peaks from those impacted by noise or background interference. A particularly important feature was coelution count, which represents the number of transitions that coelute at consistent retention times. A higher coelution count significantly boosts the confidence in identifying a valid peak. Additional features, including SNR, dot product, and shape correlation, were also considered, with their weights adjusted according to their contribution to the model's overall performance. The model was trained using cross-validation to optimize hyperparameters and prevent overfitting, ensuring that it remained robust across different datasets. This process allowed the model to generalize well, improving its ability to reliably identify peaks across a variety of samples and conditions.

This combination of manual curation and automated machine learning not only enhances the accuracy and scalability of the peak picking process but also overcomes the limitations of traditional proteomics workflows. By integrating manual validation with machine learning models trained on years of curated data, Applicants provide a more consistent, reliable, and high-throughput solution for proteomics studies. This ensures the identification of high-quality peaks across a variety of clinical samples, facilitating the analysis of large, complex datasets.

Data Preprocessing and Feature Generation: The preprocessing pipeline utilized CompleteBank-Discovered results to generate feature files, which provided a comprehensive list of candidate peaks based on observed chromatogram data. These candidate peaks were subsequently labeled according to manual validation results, with “good” peaks identified as those that met the quality criteria of coelution and high dot product scores. Features extracted from these peaks-including coelution count, dot product, signal-to-noise ratio (SNR), and shape correlation-were used as input for the XGBoost model. The model was trained to perform binary classification, distinguishing peaks as either valid or invalid based on their predicted retention times and associated feature characteristics. The model's performance in predicting accurate peak retention times and selecting the most reliable candidate peaks was evaluated using a dataset comprising over 15,000 peptides, manually validated across a variety of representative clinical samples, including pooled advanced disease plasma samples. This robust dataset ensured that the model was trained on diverse, real-world data, enhancing its accuracy and generalizability.

Peak Selection, Postprocessing, and Data Normalization: After model training, automated peak selection was applied to new, unseen data. The postprocessing steps included selecting the highest-scoring peaks for each peptide sequence, ensuring that the final selection consisted of the most reliable peaks with the highest likelihood of accurate identification. Following peak selection, data normalization was performed on the peak area data collected from each target analyte. Given the variety of detection methods and clustering based on peak intensities, hydrophobicities, retention times, and other factors, normalization was conducted for each cluster using separate methods. As a result, multiple reference points were used for normalization, tailored to each cluster of target analysis. This approach led to the development of the Multi-point Normalized Protein expression (MNPX) value, which represents the normalized expression for each protein. After normalization, the abundance of each protein across various samples could be compared, enabling the evaluation of its correlation with disease states and facilitating the identification of potential protein biomarkers. For the current study, the normalization factor for each protein was chosen as the median intensity of the biomarker's intensity in that detection cluster. Further normalization can be updated to use stably expressed plasma proteins or disease specific normalizers but will subject to further evaluation and development with at least several hundred samples for each disease type and will be updated in a further study.

### For Plasma Metabolites and Lipids

In CompletePeaking pipeline, Applicants also developed a set of metabolomics peak picking algorithms based on a second derivative method. This approach was used to identify peaks in metabolomics datasets by analyzing the intensity profiles that are changing over time.

### Smoothing the Intensity Signal

The raw intensity data was smoothed using the Savitzky-Golay filter to reduce noise while preserving peak shape. The smoothing was applied with a window length of 11 and a polynomial order of 3.

### Computing the Second Derivative

The second derivative of the smoothed intensity was computed to capture the changes in concavity that mark the boundaries of a peak. These inflection points were used to define the start and end points of each peak.

### Zero-Crossings for Boundary Detection

Zero-crossings of the second derivative were identified, marking the transition from concave up to concave down. These zero-crossings defined the start and end time points of each peak. Adjustments were made to the boundaries to account for peak asymmetry, with an extension factor applied to both the start and end points.

### Background Correction and Tail Adjustment

A background intensity threshold was calculated as the median intensity outside the peak region. The peak boundaries were iteratively adjusted to ensure that they did not extend into noise regions, and the tail symmetry condition was met to balance the peak shape.

### Width Correction

To ensure consistent peak-width estimation across samples, outliers in peak width were removed using the interquartile range (IQR) method. After removing outliers, the peak widths were standardized based on the sample with the largest total area.

This second derivative method provided a robust framework for metabolomics peak detection, and it was incorporated as an essential part of CompletePeaking pipeline to identify and quantify metabolomic signals.

### Detectability in Complete360 Methods

The Complete360 platform employs a rigorous set of criteria to ensure high-confidence detection and quantification of target proteins. These criteria were established to maximize sensitivity, reproducibility, and specificity in deep proteomic profiling. The following parameters define detectability within the Complete360 workflow:

### Signal-to-Noise Ratio (S/N)

Reliable detection of analytes requires a minimum signal-to-noise (S/N) ratio of 1.5, ensuring adequate signal intensity above background fluctuations.

### Retention Time (RT) Consistency

Retention time (RT) for analyte detection must exceed 1.5 minutes, preventing interference from early eluting compounds and maintaining consistency across runs.

### Background and Interference Control

To ensure specificity, analyte peaks must exhibit minimal interference from co-eluting species. Background signals within the retention window are required to be at least one order of magnitude lower than the analyte peak intensity. For example, an analyte peak with an intensity of 1,000 counts must have a background signal of ≤100 counts.

### Co-Elution of Fragment Ions

The apexes of monitored transitions must closely align within a time window of ±0.05-0.2 minutes, ensuring the consistency of elution profiles and preventing misidentification.

### Fragment Ion Ratio Consistency

Ion ratios between transitions of the same analyte must remain within ±20-30% of reference values, ensuring stability in detection across different analytical runs.

### Spectral Similarity Assessment

To validate fragmentation patterns, spectral comparisons with high-resolution discovery data are performed. A dot-product or similarity score of ≥0.5-0.6 is required to confirm alignment with expected fragmentation profiles.

These criteria collectively ensure the robustness and accuracy of the Complete360 methodology, facilitating the precise detection of ultra-low abundance proteins in complex biological matrices.

### Algorithm for Cross-Tumor Differential Proteomics and Enrichment Analysis

Data Acquisition and Preprocessing: The protein expression matrix (“co_data”) with rows corresponding to proteins and columns to samples across multiple disease types and normal tissues was used in this study. The protein names in row are UniProt identifiers. The columns names are sample names corresponding to the groups of Normal, Alzheimer's Disease (AD), Breast Cancer (BRCA), Colorectal Cancer (CRC), Lung Adenocarcinoma (LUAD), Ovarian Cancer (OVC), Pancreatic adenocarcinoma (PDAC), Prostate Adenocarcinoma (PRAD), and Ulcerative Colitis (UC). For the six tumor types (BRCA, CRC, LUAD, OVC, PDAC, PRAD), protein reference lists were compiled from CPTAC data or literature sources²⁶⁻³⁶. A Venn diagram was generated to show the intersection between proteins identified in “co_data” cohort and the reference lists (FIG. 8A).

Differential Expression Analysis (DEA): For each cancer type vs. normal comparison, the Wilcoxon rank-sum test (non-parametric, unpaired) was applied to identify differentially expressed proteins. Proteins were considered significantly upregulated if False Discovery Rate (FDR) <0.05 and Log₂ Fold Change (median-based)≥1. Only proteins that appeared in both the DEA results or the tumor-type-specific reference sets were retained to ensure cross-study consistency.

Heatmap Visualization of Tumor-Specific Signatures: This expression matrix was Z-score normalized by row (protein-wise) and filtered out differentially expressed proteins from each cancer type comparing to normal group. A multi-panel heatmap was generated using ‘matplotlib.gridspec’, where all the proteins differentially expressed in this cancer type (FIG. 8B) or proteins only differentially expressed in a specific cancer type (FIG. 8F) were displayed.

Functional Enrichment Analysis: Using the GSEApy/Enrichr tool, gene sets were analyzed for biological relevance. Gene set library MSigDB_Hallmark_2020 was used for cancer associated pathways. The background list of all proteins presented in the original dataset of “co_data” was provided. Significant terms (adjusted p<0.05 or <0.01, depending on results size in analysis) were extracted. The results were shown in bubble plots (FIG. 8C).

The data reported in this article have been deposited via ProteomeXchange in the PeptideAtlas SRM Experiment Library (PASSEL) (identifier PASS05916).

### REFERENCE

1. Nicholson, B. D. et al. Multi-cancer early detection test in symptomatic patients referred for cancer investigation in England and Wales (SYMPLIFY): a large-scale, observational cohort study. Lancet Oncol 24, 733-743 (2023).
2. Klein, E. A. et al. Clinical validation of a targeted methylation-based multi-cancer early detection test using an independent validation set. Ann Oncol 32, 1167-1177 (2021).
3. Cohen, J. D. et al. Detection and localization of surgically resectable cancers with a multi-analyte blood test. Science 359, 926-930 (2018).
4. Chen, M. & Zhao, H. Next-generation sequencing in liquid biopsy: cancer screening and early detection. Hum Genomics 13, 34 (2019).
5. Davies, M. P. A. et al. Plasma protein biomarkers for early prediction of lung cancer. EBioMedicine 93, 104686 (2023).
6. Wang, Q. et al. Selected reaction monitoring approach for validating peptide biomarkers. Proc Natl Acad Sci U S A 114, 13519-13524 (2017).
7. FDA (2024).
8. Vitko, D. et al. timsTOF HT Improves Protein Identification and Quantitative Reproducibility for Deep Unbiased Plasma Protein Biomarker Discovery. J Proteome Res 23, 929-938 (2024).
9. Heil, L. R. et al. Evaluating the Performance of the Astral Mass Analyzer for Quantitative Proteomics Using Data-Independent Acquisition. J Proteome Res 22, 3290-3300 (2023).
10. Bonaventura, P. et al. Identification of shared tumor epitopes from endogenous retroviruses inducing high-avidity cytotoxic T cells for cancer immunotherapy. Sci Adv 8, eabj3671 (2022).
11. Hsiue, E. H. et al. Targeting a neoantigen derived from a common TP53 mutation. Science 371 (2021 ).
12. Terai, Y. L. et al. Valid-NEO: A Multi-Omics Platform for Neoantigen Detection and Quantification from Limited Clinical Samples. Cancers (Basel) 14(2022).
13. Wang, Q. et al. Direct Detection and Quantification of Neoantigens. Cancer Immunol Res 7, 1748-1754 (2019).
14. Omenn, G. S. et al. The 2024 Report on the Human Proteome from the HUPO Human Proteome Project. J Proteome Res 23, 5296-5311 (2024).
15. Wishart, D. S. et al. MarkerDB: an online database of molecular biomarkers. Nucleic Acids Res 49, D1259-D1267 (2021).
16. Schooneveldt, Y. L. et al. The Impact of Simvastatin on Lipidomic Markers of Cardiovascular Risk in Human Liver Cells Is Secondary to the Modulation of Intracellular Cholesterol. Metabolites 11 (2021).
17. Su, B. et al. A DMS Shotgun Lipidomics Workflow Application to Facilitate High-Throughput, Comprehensive Lipidomics. J Am Soc Mass Spectrom 32, 2655-2663 (2021).
18. Cao, Z. et al. Evaluation of the Performance of Lipidyzer Platform and Its Application in the Lipidomics Analysis in Mouse Heart and Liver. J Proteome Res 19, 2742-2749 (2020).
19. Medina, J. et al. Omic-Scale High-Throughput Quantitative LC-MS/MS Approach for Circulatory Lipid Phenotyping in Clinical Research. Anal Chem 95, 3168-3179 (2023).
20. Tianqi Chen, C. G. XGBoost: A Scalable Tree Boosting System. arXiv:1603.02754 (2016).
21. Ignjatovic, V. et al. Mass Spectrometry-Based Plasma Proteomics: Considerations from Sample Collection to Achieving Translational Data. J Proteome Res 18, 4085-4097 (2019).
22. Millioni, R. et al. High abundance proteins depletion vs low abundance proteins enrichment: comparison of methods to reduce the plasma proteome complexity. PLoS One 6, e19603 (2011).
23. Tu, C. et al. Depletion of abundant plasma proteins and limitations of plasma proteomics. J Proteome Res 9, 4982-4991 (2010).
24. Douglass, J. et al. Bispecific antibodies targeting mutant RAS neoantigens. Sci Immunol 6 (2021).
25. Deutsch, E. W. et al. Advances and Utility of the Human Plasma Proteome. J Proteome Res 20, 5241-5263 (2021).
26. Li, Q. K. et al. Proteomic characterization of primary and metastatic prostate cancer reveals reduced proteinase activity in aggressive tumors. Sci Rep 11, 18936 (2021).
27. Dong, B. et al. Integrative proteogenomic profiling of high-risk prostate cancer samples from Chinese patients indicates metabolic vulnerabilities and diagnostic biomarkers. Nat Cancer 5, 1427-1447 (2024).
28. Sinha, A. et al. The Proteogenomic Landscape of Curable Prostate Cancer. Cancer Cell 35, 414-427 e416 (2019).
29. Cao, L. et al. Proteogenomic characterization of pancreatic ductal adenocarcinoma. Cell 184, 5031-5052 e5026 (2021).
30. Hu, Y. et al. Integrated Proteomic and Glycoproteomic Characterization of Human High-Grade Serous Ovarian Carcinoma. Cell Rep 33, 108276 (2020).
31. Zhang, H. et al. Integrated Proteogenomic Characterization of Human High-Grade Serous Ovarian Cancer. Cell 166, 755-765 (2016).
32. Gillette, M. A. et al. Proteogenomic Characterization Reveals Therapeutic Vulnerabilities in Lung Adenocarcinoma. Cell 182, 200-225 e235 (2020).
33. Krug, K. et al. Proteogenomic Landscape of Breast Cancer Tumorigenesis and Targeted Therapy. Cell 183, 1436-1456 e1431 (2020).
34. Mertins, P. et al. Proteogenomics connects somatic mutations to signalling in breast cancer. Nature 534, 55-62 (2016).
35. Vasaikar, S. et al. Proteogenomic Analysis of Human Colon Cancer Reveals New Therapeutic Opportunities. Cell 177, 1035-1049 e1019 (2019).
36. Zhang, B. et al. Proteogenomic characterization of human colon and rectal cancer. Nature 513, 382-387 (2014).
37. Pettersson, F. et al. Ribavirin treatment effects on breast cancers overexpressing eIF4E, a biomarker with prognostic specificity for luminal B-type breast cancer. Clin Cancer Res 17, 2874-2884 (2011).
38. Gao, Y. et al. Loss of ERalpha induces amoeboid-like migration of breast cancer cells by downregulating vinculin. Nat Commun 8, 14483 (2017).
39. Ding, Q. et al. Ficolin-2 triggers antitumor effect by activating macrophages and CD8(+) T cells. Clin Immunol 183, 145-157 (2017).
40. Cao, B. et al. Cancer-mutated ribosome protein L22 (RPL22/eL22) suppresses cancer cell survival by blocking p53-MDM2 circuit. Oncotarget 8, 90651-90661 (2017).
41. Niu, L. et al. Plasma proteome variation and its genetic determinants in children and adolescents. Nat Genet (2025).
42. Obradovic, M. et al. Leptin and Obesity: Role and Clinical Implication. Front Endocrinol (Lausanne) 12, 585887 (2021).
43. Candia, J. et al. Assessment of Variability in the SOMAscan Assay. Sci Rep 7, 14248 (2017).
44. Candia, J., Daya, G. N., Tanaka, T., Ferrucci, L. & Walker, K. A. Assessment of variability in the plasma 7k SomaScan proteomics assay. Sci Rep 12, 17147 (2022).
45. Smits, H. M. et al. The BAMBOO method for correcting batch effects in high throughput proximity extension assays for proteomic studies. Sci Rep 15, 1498 (2025).
46. Dennis, M. S. et al. Albumin binding as a general strategy for improving the pharmacokinetics of proteins. J Biol Chem 277, 35035-35043 (2002).
47. Hui, H., Farilla, L., Merkel, P. & Perfetti, R. The short half-life of glucagon-like peptide-1 in plasma does not reflect its long-lasting beneficial effects. Eur J Endocrinol 146, 863-869 (2002).
48. Razavi, M. et al. Measuring the Turnover Rate of Clinically Important Plasma Proteins using an Automated SISCAPA Workflow. Clin Chem 65, 492-494 (2019).
49. Wang, Y. et al. Reversed-phase chromatography with multiple fraction concatenation strategy for proteome profiling of human MCF10A cells. Proteomics 11, 2019-2026 (2011).
50. Demichev, V., Messner, C. B., Vernardis, S. I., Lilley, K. S. & Ralser, M. DIA-NN: neural networks and interference correction enable deep proteome coverage in high throughput. Nat Methods 17, 41-44 (2020).
51. Kong, A. T., Leprevost, F. V., Avtonomov, D. M., Mellacheruvu, D. & Nesvizhskii, A. I. MSFragger: ultrafast and comprehensive peptide identification in mass spectrometry-based proteomics. Nat Methods 14, 513-520 (2017).

1. A method for automated peak picking in multi-omics biological sample data comprising:
a. preprocessing biological sample data to generate feature files containing candidate peaks for target analytes;
b. extracting features from said feature files, including coelution metrics, spectral similarity measures, signal quality parameters, and intensity values;
c. training a machine learning model on manually curated data to predict high-quality peaks, wherein said model learns to distinguish valid peaks from noise and interference;
d. applying the trained model to new datasets to predict peak quality for each candidate peak;
e. postprocessing predictions to retain the most reliable peaks for each target analyte.

2. A system for automated peak picking in multi-omics data comprising:
a. a data preprocessing module configured to generate feature files from biological sample data;
b. a feature extraction module configured to extract and label analytical features from said feature files;
c. a machine learning module configured to train a predictive model using extracted features and manual validation labels;
d. a postprocessing module configured to select highest-scoring predictions for each target analyte.

3. The method of claim 1, wherein the machine learning model comprises an XGBoost gradient boosting framework optimized through cross-validation.

4. The method of claim 1, wherein the biological sample data comprises plasma proteomics, metabolomics, and lipidomics data analyzed simultaneously.

5. The method of claim 1, wherein the features include coelution count, dot product score, signal-to-noise ratio, shape correlation, and peak intensity.

6. The method of claim 1, wherein the biological sample data comprises Selected Reaction Monitoring (SRM) or Multiple Reaction Monitoring (MRM) proteomics data with mProphet feature file generation.

7. The method of claim 6, wherein the coelution metrics comprise counting transitions that coelute within a defined retention time window, and
wherein spectral similarity measures comprise dot product calculations comparing observed peaks to reference library spectra.

8. The method of claim 1, wherein the biological sample data comprises metabolomics data and the method further comprises:
a. smoothing intensity signals using Savitzky-Golay filtering;
b. computing second derivatives of smoothed intensity data;
c. identifying zero-crossings of the second derivative to define peak boundaries;
d. applying background correction and width standardization using interquartile range methods.

9. The method of claim 8, wherein peak boundaries are adjusted using extension factors to account for peak asymmetry.

10. The method of claim 1, wherein the biological sample data comprises blood or plasma samples, and
wherein the method utilizes consistent noise patterns from blood matrix components as reproducible landmarks for enhanced peak identification without requiring exogenous internal standards.

11. The method of claim 10, wherein the noise patterns originate from high-abundance proteins, lipids, or other prevalent blood substances that provide consistent reproducibility across different specimens.

12. The method of claim 1, further comprising normalizing peak area data using Multi-point Normalized Protein eXpression (MNPX) values, wherein normalization is performed separately for different analytical clusters based on peak intensities, hydrophobicities, and retention times.

13. The method of claim 12, wherein multiple reference points are used for normalization, tailored to each cluster of target analysis.

14. The method of claim 1, wherein the manually curated data comprises samples from patients with cancer, cardiovascular disease, neurodegenerative disease, or autoimmune disease for training disease-specific pattern recognition.

15. The method of claim 1, wherein the target analytes comprise proteins, metabolites, and lipids simultaneously analyzed from the same biological sample for detecting disease-specific molecular signatures.

16. The method of claim 1, wherein reliable detection requires a signal-to-noise ratio of at least 1.5, retention time exceeding 1.5 minutes, and background signals at least one order of magnitude lower than analyte peak intensity.

17. The method of claim 1, wherein fragment ion ratios between transitions remain within ±20-30% of reference values and wherein spectral similarity scores exceed 0.5-0.6 for validation.

18. The system of claim 2, wherein the machine learning module implements XGBoost with hyperparameters optimized through grid search and cross-validation, and further comprising a normalization module configured to apply Multi-point Normalized Protein expression (MNPX) values.

19. A method of disease diagnosis comprising:
a. obtaining a blood or plasma sample from a subject;
b. analyzing said sample using the method of claim 1 to identify molecular signatures comprising proteins, metabolites, and lipids;
c. comparing identified signatures to disease-specific reference patterns;
d. determining disease status based on signature comparison.

20. The method of claim 19, wherein the molecular signatures are associated with cancer, cardiovascular disease, neurodegenerative disease, or autoimmune disease, and wherein the analysis provides simultaneous multi-omics characterization from a single sample.
